(19) United States
(12) Patent Application Publication
(10) Pub. No.: US 2005/0288375 A1
(43) Pub. Date: Dec. 29, 2005

(54) Method and composition for treating neurodegenerative disorders

(75) Inventors: Adrian Hobden, Salt Lake City, UT (US); Kenton Zavitz, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/197,942

(22) Filed: Aug. 5, 2005

Related U.S. Application Data
(63) Continuation of application No. PCT/US04/03618, filed on Feb. 5, 2004.
(60) Provisional application No. 60/445,587, filed on Feb. 5, 2003.
(60) Provisional application No. 60/448,914.
(60) Provisional application No. 60/495,233, filed on Aug. 13, 2003.

(51) Int. Cl. A61K 31/192; A61K 31/13

(52) U.S. Cl. 514/569; 514/570; 514/663

(57) ABSTRACT

The invention provides compositions and methods for treating neurodegenerative disorders. A method of the invention involves administering to an individual in need of treatment a composition having an R-NSAID and an NMDA antagonist. Another method of the invention involves administering to an individual in need of treatment a composition having at least two compounds that are capable of interacting with CYP2C9, wherein at least one of said compounds is an Aβ₄₂ lowering agent. The methods and compositions of the invention are useful for treating and preventing neurodegenerative disorders like Alzheimer's disease, dementia, mild cognitive impairment.

### RELATED APPLICATIONS

This application is a continuation-in-part of PCT Application No. PCT/US04/03618 filed on Feb. 5, 2004, which is related to U.S. Provisional Application Ser. No. 60/448,914 filed on Feb. 21, 2003, U.S. Provisional Application Ser. No. 60/445,587 filed on Feb. 5, 2003, and U.S. Provisional Application Ser. No. 60/495,233 filed on Aug. 13, 2003, which are incorporated herein by reference in their entirety.

### FIELD OF THE INVENTION

The invention provides compositions and methods for the therapeutic treatment of neurodegenerative disorders. The invention provides a composition having an R-NSAID and a NMDA antagonist. The invention provides a method for treating neurodegenerative disorders through the administration of an R-NSAID and a NMDA antagonist. The invention also provides compositions useful for the prevention and/or treatment of neurodegenerative diseases and having at least two compounds that are capable of interacting with the cytochrome P450 enzyme CYP2C9, wherein at least one of the compounds is an amyloid β₄₂ protein (Aβ₄₂) lowering agent. The invention also provides a method for treating neurodegenerative disorders through the administration of at least two compounds that are capable of interacting with the cytochrome P450 enzyme CYP2C9, wherein at least one of the compounds is an Aβ₄₂ lowering agent. The invention is useful for treating and preventing neurodegenerative disorders such as Alzheimer's disease, dementia, and mild cognitive impairment.

### BACKGROUND OF THE INVENTION

Dementia is a brain disorder that seriously affects a person's ability to carry out normal daily activities. Among older people, Alzheimer's disease (AD) is the most common form of dementia and involves parts of the brain that control thought, memory, and language. Despite intensive research throughout the world, the causes of AD are still unknown and there is no cure. AD most commonly begins after the age of 60, with the risk of acquiring the disease increasing with age. Younger people can also get AD, but it is much less common. It is estimated that 3 percent of men and women ages 65 to 74 have AD. Almost half of those ages 85 and older may have the disease. AD is not a normal part of aging. Alzheimer's disease is a complex disease that can be caused by genetic and environmental factors.

In 1906, Dr. Alois Alzheimer noticed changes in the brain tissue of a woman who had died of an unusual mental illness. In her brain tissue, he found abnormal clumps (now known as amyloid plaques) and tangled bundles of fibers (now known as neurofibrillary tangles) which, today, are considered the pathological hallmarks of AD. Other brain changes in people with AD have been discovered. For example, with AD, there is a loss of nerve cells in areas of the brain that are vital to memory and other mental abilities. Scientists have also found that there are lower levels of chemicals in the brain that carry complex messages back and forth between nerve cells. AD may disrupt normal thinking and memory by blocking these messages between nerve cells.

Plaques and tangles are found in the same brain regions that are affected by neuronal and synaptic loss. Neuronal and synaptic loss is universally recognized as the primary cause of decline in cognitive function in AD patients. The number of tangles is more highly correlated with cognitive decline than amyloid load in patients with AD (Albert PNAS 93:13547-13551 (1996)). The cellular, biochemical, and molecular events responsible for neuronal and synaptic loss in AD are not known. A number of studies have demonstrated that amyloid can be directly toxic to neurons resulting in behavioral impairment (Iversen et al. Biochem. J. 311:1-16 (1995); Weiss et al. J. Neurochem. 62:372-375 (1994); Lorenzo et al. Ann N Y Acad. Sci. 777:89-95 (1996); Storey et al. Neuropathol. Appl. Neurobiol. 2:81-97 (1999)). The toxicity of amyloid or tangles is potentially aggravated by activation of the complement cascade (Rogers et al. PNAS 21:10016-10020 (1992); Rozemuller et al. Res. Immunol. 6:646-9 (1992); Rogers et al. Res Immunol. 6:624-30 (1992); Webster et al. J. Neurochem. 69(1):388-98 (1997)). This suggests involvement of an inflammatory process in AD and neuronal death seen in AD (Fagarasan et al. Brain Res. 723(1-2):231-4. (1996); Kalaria et al. Neurodegeneration. 5(4):497-503 (1996); Kalaria et al. Neurobiol Aging. 17(5):687-93 (1996); Farlow Am J Health Syst Pharm. 55 Suppl. 2:S5-10 (1998).

Evidence that amyloid β protein (Aβ) deposition causes some forms of AD was provided by genetic and molecular studies of some familial forms of AD (FAD). (See, e.g., Ii Drugs Aging 7(2):97-109 (1995); Hardy PNAS 94(6):2095-7 (1997); Selkoe J. Biol. Chem. 271(31):18295-8 (1996)). The amyloid plaque buildup in AD patients suggests that abnormal processing of Aβ may be a cause of AD. Aβ is a peptide of 39 to 42 amino acids and is the core of senile plaques observed in all Alzheimer's disease cases. If abnormal processing is the primary cause of AD, then familial Alzheimer's disease (FAD) mutations that are linked (genetically) to FAD may induce changes that, in one way or another, foster Aβ deposition. There are 3 FAD genes known so far (Hardy et al. Science 282:1075-9 (1998); Ray et al. (1998)). Mutations in these FAD genes can result in increased Aβ deposition. It is noted that the vast majority of Alzheimer's disease cases are not a result of mutations in FAD genes.

The first of the 3 FAD genes codes for the Aβ precursor, amyloid precursor protein (APP) (Selkoe J. Biol. Chem. 271(31):18295-8 (1996)). Mutations in the APP gene are very rare, but all of them cause AD with 100% penetrance and result in elevated production of either total Aβ or Aβ₄₂, both in model transfected cells and transgenic animals. The other two FAD genes code for presenilin 1 and 2 (PS1, PS2) (Hardy PNAS 94(6):2095-7 (1997)). The presenilins contain 8 transmembrane domains and several lines of evidence suggest that they are involved in intracellular protein trafficking. Other studies suggest that the presenilins function as proteases. Mutations in the presenilin genes are more common than in the APP genes, and all of them also cause FAD with 100% penetrance. Similar to APP mutants, studies have demonstrated that PS1 and PS2 mutations shift APP metabolism, resulting in elevated Aβ₄₂ production (in vitro and in vivo).

Cycloxygenases (COX) are major Alzheimer's disease drug targets due to the epidemiological association of NSAID use, whose primary target are cycloxygenases, with a reduced risk of developing Alzheimer's disease (see, e.g., Hoozemans et al. Curr. Drug Targets 4(6):461-8 (2003) and Pasinetti et al. J. Neurosci. Res. 54(1):1-6 (1998)). The epidemiological studies have indicated that chronic NSAID use appears to reduce the risk of acquiring Alzheimer's disease and/or delay the onset of the disease. COX-2 selective inhibitors are attractive candidates for long-term drug use since they do not inhibit COX-1 and appear to be less-toxic. In support of COX-2 being a target for treating AD, a recent study was published reporting that in mouse models of AD, COX-2 overexpression was related to the neuropathology of AD (Xiang et al. Neurobiol. Aging 23:327-34 (2002)). At the 8^{th} international conference on Alzheimer's disease and related disorders, it was reported that rofecoxib, a COX-2 selective NSAID, at 25 mg daily, failed to show efficacy for treating AD. Naproxen, a nonselective COX inhibitor, in the same trial failed to show efficacy in Alzheimer's disease treatment. See Aisen et al. (JAMA 289:2819-26 (2003)). These authors concluded that the results with naproxen and rofecoxib do not support the use of NSAIDS for the treatment of AD.

Aβ formation is another target for affecting Alzheimer's disease progression since Aβ amyloid plaques are a central pathological hallmark of the disease. Recently, it was suggested that certain NSAIDs are capable of lowering the level of Aβ₄₂. U.S. Patent Application 2002/0128319 to Koo et al. discloses the use of an Aβ₄₂ lowering amount of NSAID for treating Alzheimer's disease. The hope is that by lowering the level of Aβ₄₂, the formation of the amyloid plaques central to the disease would be retarded. Interestingly, several studies have pointed to a link between amyloid plaque formation and COX-2 overexpression (see, e.g., Xiang et al. Gene Expr. 10(5-6):271-8 (2002)).

A recent clinical trial using a therapy designed to eliminate Aβ plaques from disease patients failed despite strong evidence of efficacy in animal models (Pieffer et al. Science 298:1379 (2002)). The Aβ-lowering therapy that worked in animal models caused serious problems in humans. In view of the clinical studies, Atwood et al. (Science 299:1014 (2003)) noted that “Mounting evidence indicates that this deposition of amyloid-β may be a neuroprotective response to injury” and “These results demonstrate yet again the futility of removing a protein, amyloid-β, which has ubiquitous tissue expression, without first understanding its function(s).” Additionally, secretase inhibitors, which were designed to alter processing of APP, have turned out to be toxic compounds not likely to be suitable for chronic human use. Thus, it is not clear if reducing Aβ or Aβ₄₂ is a realistic treatment/prevention option. Indeed, as noted recently, mutations in PS-1 associated with AD may cause the disease not through altering Aβ processing but rather by affecting calcium homeostasis (Mattson Nature 442:385-386 (2003)).

Several epidemiological studies have reported an association between long-term use of NSAIDs, such as ibuprofen and aspirin, with reduced risk for certain malignancies and neurodegenerative processes characterized by dementia of the Alzheimer's type. A variety of explanations have been given for the reduced cancer and Alzheimer's disease (AD) risk associated with long-term NSAID use. The primary action of NSAIDs appears to be inhibition of cyclooxygenase (COX) activity. Thus, a leading hypothesis is that NSAIDs reduce risk for certain cancers and Alzheimer's disease by affecting the COX enzymes. Other explanations include mediation of apoptosis, modulation of growth factors, and modulation of the nuclear factor kappa B pathway (NF-κB).

U.S. Pat. No. 5,192,753 to McGeer et al. discloses the use of NSAIDs to treat Alzheimer's disease through the inhibition of cyclooxygenase and therefore inhibition of prostaglandin synthesis. U.S. Pat. Nos. 5,643,960 and 6,025,395 both to Brietner et al. disclose the use of COX inhibiting NSAIDs to delay the onset of Alzheimer's disease. Despite the incredible wealth of information regarding NSAID use and its link to a reduced risk of developing Alzheimer's disease, there is no Food and Drug Administration (FDA) approved NSAID indication for Alzheimer's disease or any other equivalent national approval agency. Furthermore, several promising NSAIDs have failed in clinical trial designed to test their efficacy in treating AD.

In the United States alone, four million adults suffer from Alzheimer's disease (AD). Not only is Alzheimer's disease significantly impacting the lives of countless families today, it is threatening to become even more of a problem as the baby boom generation matures. The economic burden of AD is estimated to cost over $100 billion a year and the average lifetime cost per patient is estimated to be $174,000.

Unfortunately, there is no cure available for AD. Of the five drugs currently being used in the US for the treatment of AD, four of them—tacrine (Cognex®), donepezil (Aricept®), rivastigmine (Exelon®), and galantamine (Reminyl®), are inhibitors of acetylcholine esterase. Another drug, memantine, was recently approved for treating moderate-to-severe AD. More recently it was reported that memantine showed efficacy in treating mild-to-moderate AD. Memantine is a NMDA receptor antagonist.

NMDA receptors have been studied extensively. NMDA receptors are known mediate synaptic transmission and neural plasticity in the mammalian central nervous system. (See, Monaghan Annu Rev Pharmacol Toxicol, 29:365-402 (1989); Collingridge Pharmacol Rev, 41:143-210 (1989); McBain Physiol Rev, 74:723-60(1994)). NMDA receptors are differentially expressed during development (Sheng Nature, 368:144-7 (1994)). NMDA receptors are involved in a variety of fundamental biological processes including brain development by stabilizing converging synapses (Scheetz Faseb J, 8:745-52 (1994)), stimulating cerebellar granule cell migration (Hitoshi et al., Science, 260:95-97 (1993); Farrant Nature, 368:335-9 (1994); Rossi Neuropharmacology, 32:1239-48 (1993)) and development (Burgoyne J Neurocytol, 22:689-95 (1993)), inducing long term depression (Battistin Eur J Neurosci, 6:1750-5 (1994); Komatsu Neuroreport, 4:907-10 (1993); Tsumoto Jpn J. Physiol., 40:573-93 (1990)) and apoptosis (Finiels J Neurochem, 65:1027-34 (1995); Ankarcrona FEBS Lett, 394:321-4 (1996)). NMDA receptors are also known contribute to excitatory cell death in a number of adult pathological conditions (Greenamyre Neurobiol Aging, 10:593-602 (1989); Meldrum Trends Pharmacol Sci, 11, (1990) 379-87; Clark, S, “The NMDA receptor in epilepsy”, 2 edn., Oxford University Press, Oxford, 1994, 395-427 pp.; Doble, A., Therapie, 50:319-37 (1995)).

Excitatory amino acid receptors, including NMDA receptors, are known to be involved in neurodegenerative diseases, and specific NMDA antagonists are being used in clinical research (Lipton Trends Neurosci, 16:527-32 (1993)) for the potential treatment of stroke, CNS trauma (Faden Trends Pharmacol Sci, 13:29-35 (1992)), epilepsy (Thomas J Am Geriatr Soc, 43:1279-89 (1995); Perucca Pharmacol Res, 28:89-106 (1993)), pain (Elliott Neuropsychopharmacology, 13:347-56 (1995)), Huntington's disease (Purdon J Psychiatry Neurosci, 19:359-67 (1994)), AIDS dementia (Lipton Dev Neurosci, 16:145-51 (1994); Lipton Ann N Y Acad Sci, 747:205-24 (1994)), and Alzheimer's disease (Barry Arch Phys Med Rehabil, 72:1095-101 (1991)) and Parkinson's disease (Ossowska N Neural Transm Park Dis Dement Sect, 8:39-71 (1994)) (Rogawski Trends Pharmacol Sci, 14:325-31 (1993)). In vivo treatment with some of these agents manifest PCP-like psychotomimetic effects. Hence, research has been underway to discover and develop more therapeutically useful and less toxic drugs (Willetts Trends Pharmacol Sci, 11:423-8 (1990)). One less-toxic NMDA antagonist candidate is Ro-01-6794/706 or dextrorphan (Ann N Y Acad Sci, 765 249-61, 298 (1995)). Dextromethorphan and it's metabloite dextrorphan are widely used over the counter as antitussives (Irwin Drugs, 46:80-91 (1993)) which are NMDA channel blockers (Fekany Eur J Pharmacol, 151:151-4 (1988); Choi J Pharmacol Exp Ther, 242:713-20 (1987)) that may be a clinically useful neuroprotectant (Steinberg Neurosci Lett 133:225-8 (1991)). Therapeutically tolerated doses of roughly 30 mg (q.i.d.) orally are used for the over the counter antitussive action, and to 90 mg (q.i.d.) orally for clinical treatment of brain ischemia (Albers Clin. Neuropharmacol., 15:509-14 (1992)). Side effects at high doses of dextromethorphan and dextrorphan included drowsiness, nausea, and decreased coordination. Toxic high doses of dextromethorphan and dextrorphan have been described (Wolfe Am J Emerg Med, 13:174-6 (1995); Hinsberger J Psychiatry Neurosci, 19:375-7(1994)); Loscher Eur J Pharmacol, 238:191-200 (1993)).

Numerous potentially clinically useful NMDA antagonists have been studied (Jane “Agonists and competitive antagonists: structure-activity and molecular modeling studies”, 2 edn., Oxford University Press, Oxford, 1994, 31-104 pp; Andaloro Society for Neuroscience Abstracts, 604 (1996); Bigge Biochem Pharmacol, 45:1547-61 (1993); Ornstein, P., “The development of novel competitive N-methyl-D-aspartate antagonists as useful therapeutic agents: Discovery of LY274614 and LY233536”, Raven Press, New York, 1991, 415-423 pp), and some are even orally available, including some derivatives EAB-515 (Li J Med Chem, 38 1955-65 (1995); Lowe Neurochem Int, 25:583-600 (1994)), memantine (Parsons Neuropharmacology, 34:1239-58 (1995); Kornhuber J Neural Transm Suppl, 43:91-104 (1994); Wenk Eur J Pharmacol, 293 267-70 (1995)), and ketamine (Parsons Neuropharmacology, 34:1239-58 (1995); Sagratella Pharmacol Res, 32:1-13 (1995); Porter J Neurochem, 64:614-23 (1995)). Some of these NMDA antagonists are approved for use, several others are in clinical trials for the treatment of neurodegenerative disease, epilepsy, stroke, and other diseases.

References which disclose other NMDA receptor blockers as well as assays for identifying an agent that acts as such a blocker and toxicity studies for pharmacologic profiles are disclosed in the foregoing and following articles which are all hereby incorporated by reference in their entirety. (See also Jia-He Li, et al., J Med Chem 38:1955-1965 (1995); Steinberg et al., Neurosci Lett, 133:225-8 (1991); Meldrum et al., Trends Pharmacol Sci, 11:379-87 (1990); Willetts et al., Trends Pharmacol Sci, 11:423-8 (1990); Faden et al., Trends Pharmacol Sci, 13:29-35 (1992); Rogawski, Trends Pharmacol Sci, 14:325-31 (1993); Albers et al, Clinical Neuropharm, 15:509-514 (1992); Wolfe et al., Am J Emerg Med, 13:174-6 (1995); Bigge, Biochem Pharmacol, 45:1547-61 (1993)).

The drugs currently used for treating AD, including memantine and the acetylcholine esterase inhibitors, are marginally efficacious and have undesirable side-effects. Thus, there is a large unmet need for better and safer drugs.

### SUMMARY OF THE INVENTION

The invention generally relates to compositions and therapeutic treatments for neurodegenerative disorders. More specifically, the invention provides a composition for treating and preventing neurodegenerative disorders. One composition of the invention has at least one NMDA antagonist (N-methyl-D-aspartate) and at least one R-NSAID (non-steroidal anti-inflammatory) and at least one pharmaceutically acceptable carrier. Another composition of the invention comprises two compounds that are capable of interacting with CYP2C9, wherein at least one of the compounds is an Aβ₄₂ lowering agent. One method of the invention involves treating an individual in need of treatment (or prophylaxis) with a neurodegenerative disease treating (or prophylactic) effective amount of at least one NMDA antagonist and at least one R-NSAID. Another method of the invention involves treating an individual in need of treatment with a therapeutically or prophylactically effective amount of at least two compounds that are capable of interacting with CYP2C9, wherein at least one of the compounds is an Aβ₄₂ lowering agent.

In a first embodiment, the invention provides a composition comprising at least one NMDA antagonist and at least one R-NSAID. In one aspect of this embodiment, the at least one NMDA antagonist is selected from the group consisting of memantine, adamantane, amantadine, an adamantane derivative, dextromethorphan, dextrorphan, dizocilpine, ibogaine, ketamine, remacemide, and phencyclidine. In another aspect of this embodiment the at least one NMDA antagonist is memantine. In one aspect of this embodiment the R-NSAID is selected from the group consisting of R-flurbiprofen, R-ketoprofen, R-ketorolac, R-naproxen, R-tiaprofenic acid, R-suprofen, R-carprofen, R-pirprofen, R-indoprofen, R-benoxaprofen, and R-etolodac. In yet another aspect of this embodiment the NMDA antagonist is selected from the group consisting of memantine, adamantane, amantadine, an adamantane derivative, dextromethorphan, dextrorphan, dizocilpine, ibogaine, ketamine, remacemide, and phencyclidine, and the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. In still another aspect of this embodiment, the R-NSAID is R-flurbiprofen. In another aspect, the R-NSAID is R-flurbiprofen and the NMDA antagonist is selected from the group consisting of memantine, adamantane, amantadine, an adamantane derivative, dextromethorphan, dextrorphan, dizocilpine, ibogaine, ketamine, remacemide, and phencyclidine. The invention further provides compositions having R-flurbiprofen and memantine; R-flurbiprofen and adamantane; R-flurbiprofen and amantadine; R-flurbiprofen and an adamantane derivative; R-flurbiprofen and dextromethorphan; R-flurbiprofen and dextrorphan; R-flurbiprofen and dizocilpine; R-flurbiprofen and ibogaine; R-flurbiprofen and ketamine; R-flurbiprofen and remacemide; and R-flurbiprofen and phenylcyclidine. The compositions of this embodiment can provide the two components together in a single dose with a pharmaceutically acceptable carrier.

In a second embodiment, the invention provides compositions comprising two compounds that are capable of interacting with CYP2C9, wherein at least one of the compounds is an Aβ₄₂ lowering agent. In one aspect of this embodiment, one of the compounds is a substrate of CYP2C9. In another aspect of this embodiment, one of the compounds is a CYP2C9 inhibitor. In another aspect of this embodiment, one compound is a CYP2C9 substrate and another compound is a CYP2C9 inhibitor. In yet another aspect of this embodiment, one of the compounds is an NSAID (preferably an R-NSAID), a modified NSAID, an NSAID derivative, or NSAID analogue. In still another aspect of this embodiment, one of the compounds is a statin or a derivative or analogue of a statin. In another embodiment one of the compounds is capable of lowering Aβ₂ levels and increasing Aβ₃₈ levels, while not affecting Aβ₄₀ levels. The composition of this embodiment can also increase the levels of other Aβ proteins smaller than Aβ₄₀, including Aβ₃₄, Aβ₃₇, Aβ₃₈, and Aβ₃₉.

In a third embodiment, the invention provides a method for treating neurodegenerative disorders. According to the method of this embodiment, an effective amount of at least one R-NSAID and at least one NMDA antagonist is administered to an individual in need of such treatment. The individual in need of treatment can have a neurodegenerative disorder, a predisposition to a neurodegenerative disorder, and/or desire prophylaxis against neurodegenerative disorders. In one aspect of this embodiment, the effective amount of the at least one R-NSAID and at least one NMDA antagonist is capable of reducing at least one symptom of the neurodegenerative disorder. In another aspect, for individuals desiring prophylaxis against a neurodegenerative disorder, the effective amount of the at least one R-NSAID and at least one NMDA antagonist, is capable of preventing an increase (or rate of increase) in at least one symptom of the neurodegenerative disorder. For example, the treatment can slow the rate of cognitive decline. In one aspect of this method, the at least one NMDA antagonist is memantine. In another aspect of this method, the at least one NMDA antagonist is selected from the group consisting of memantine, adamantane, amantadine, an adamantane derivative, dextromethorphan, dextrorphan, dizocilpine, ibogaine, ketamine, remacemide, and phencyclidine. In one aspect of this method, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. In yet another aspect of this method, the NMDA antagonist is selected from the group consisting of memantine, adamantane, amantadine, an adamantane derivative, dextromethorphan, dextrorphan, dizocilpine, ibogaine, ketamine, remacemide, and phencyclidine, and the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. In still another aspect of this method, the R-NSAID is R-flurbiprofen. In another aspect of this method the R-NSAID is R-flurbiprofen and the NMDA antagonist is selected from the group consisting of memantine, adamantane, amantadine, an adamantane derivative, dextromethorphan, dextrorphan, dizocilpine, ibogaine, ketamine, remacemide, and phencyclidine. The method of the invention further provides for the treatment or prophylaxis of neurodegenerative disorders by administering an effective amount of R-flurbiprofen and memantine; R-flurbiprofen and adamantane; R-flurbiprofen and amantadine; R-flurbiprofen and an adamantane derivative; R-flurbiprofen and dextromethorphan; R-flurbiprofen and dextrorphan; R-flurbiprofen and dizocilpine; R-flurbiprofen and ibogaine; R-flurbiprofen and ketamine; R-flurbiprofen and ketamine; R-flurbiprofen and remacemide; or R-flurbiprofen and phenylcyclidine. In a preferred aspect of this method, the neurodegenerative disease is selected from the group consisting of Alzheimer's disease, dementia, and mild cognitive impairment. In another preferred embodiment, the invention provides a method for the treatment or prophylaxis of Alzheimer's disease through the administration of an Alzheimer's disease treating or prophylactic effective amount of R-flurbiprofen and memantine; R-flurbiprofen and adamantane; R-flurbiprofen and an adamantane derivative; R-flurbiprofen and dextromethorphan; R-flurbiprofen and dextrorphan; R-flurbiprofen and dizocilpine; R-flurbiprofen and ibogaine; R-flurbiprofen and ketamine; R-flurbiprofen and remacemide; or R-flurbiprofen and phenylcyclidine.

In a fourth embodiment, the invention provides a method for treating or preventing neurodegenerative disorders such as Alzheimer's disease. In particular, this method relates to treating or delaying the onset of neurodegenerative disorders by administering to an individual a therapeutically or prophylactically effective amount of at least two compounds that are capable of interacting with CYP2C9, wherein at least one of the compounds is an Aβ₄₂ lowering agent. This method may treat (or slow the onset of the progression of) the disease or disorder. This method may also be used to delay or slow the onset of the disease or disorder or signs or symptoms thereof.

In a fifth embodiment, the invention provides a method of reducing (or reducing the rate of increase of) amyloid β42 (Aβ₄₂) protein levels. In particular, the method relates to reducing, lowering, or preventing an increase in Aβ₄₂ protein levels, in an individual in need of such treatment, by administering to the individual an effective amount of at least one R-NSAID and at least one NMDA antagonist. The individual in need of treatment can have a neurodegenerative disorder, a predisposition to a neurodegenerative disorder, and/or desire prophylaxis against neurodegenerative disorders, where the disorder is characterized by increased Aβ₄₂ protein levels (or abnormal APP processing). In one aspect, the effective amount is an amount of at least one R-NSAID and at least one NMDA antagonist sufficient for reducing Aβ₄₂ protein levels. In another aspect, for individuals desiring prophylaxis against a neurodegenerative disorder, the effective amount is an amount of at least one R-NSAID and at least one NMDA antagonist, sufficient for preventing an increase in Aβ₄₂ protein levels or an increase in the rate of Aβ₄₂ increase. In one aspect of this method, the at least one NMDA antagonist is memantine. In another aspect of this method, the at least one NMDA antagonist is selected from the group consisting of memantine, adamantane, amantadine, an adamantane derivative, dextromethorphan, dextrorphan, dizocilpine, ibogaine, ketamine, remacemide, and phencyclidine. In one aspect of this method, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. In yet another aspect of this method, the NMDA antagonist is selected from the group consisting of memantine, adamantane, amantadine, an adamantane derivative, dextromethorphan, dextrorphan, dizocilpine, ibogaine, ketamine, remacemide, and phencyclidine, and the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. In still another aspect of this method, the R-NSAID is R-flurbiprofen. In another aspect of this method, the R-NSAID is R-flurbiprofen and the NMDA antagonist is selected from the group consisting of memantine, adamantane, amantadine, an adamantane derivative, dextromethorphan, dextrorphan, dizocilpine, ibogaine, ketamine, remacemide, and phencyclidine. The method of the invention further provides for the treatment or prophylaxis of neurodegenerative disorders with an Aβ₄₂ protein lowering effective amount of R-flurbiprofen and memantine; R-flurbiprofen and adamantane; R-flurbiprofen and amantadine; R-flurbiprofen and an adamantane derivative; R-flurbiprofen and dextromethorphan; R-flurbiprofen and dextrorphan; R-flurbiprofen and dizocilpine; R-flurbiprofen and ibogaine; R-flurbiprofen and ketamine; R-flurbiprofen and remacemide; or R-flurbiprofen and phenylcyclidine. In a preferred aspect of this method, the neurodegenerative disease is selected from the group consisting of Alzheimer's disease, dementia, and mild cognitive impairment. In another preferred embodiment, the invention provides a method for the treatment or prophylaxis of Alzheimer's disease through the administration of an Aβ₄₂ protein lowering effective amount of R-flurbiprofen and memantine; R-flurbiprofen and adamantane; R-flurbiprofen and an adamantane derivative; R-flurbiprofen and dextromethorphan; R-flurbiprofen and dextrorphan; R-flurbiprofen and dizocilpine; R-flurbiprofen and ibogaine; R-flurbiprofen and ketamine; R-flurbiprofen and ketamine; R-flurbiprofen and remacemide; or R-flurbiprofen and phenylcyclidine.

In a sixth embodiment, the invention provides a method of reducing Aβ₄₂ protein levels in an individual. In particular, this method relates to reducing, lowering, or preventing an increase in Aβ₄₂ levels in an individual by administering to the individual a therapeutically or prophylactically effective amount of at least two compounds that are capable of interacting with CYP2C9, wherein at least one of the compounds is an Aβ₄₂ lowering agent. This method may treat (or prevent the progression of) Aβ₄₂ related diseases or disorders. This method may also slow the onset (or rate of increase) of signs or symptoms of the disease or disorder. In a preferred aspect of this embodiment, the administered compounds lower Aβ₄₂ levels to a greater extent than they inhibit COX-1, COX-2, or a combination thereof. In yet another aspect of this embodiment, the invention provides a method of lowering Aβ₄₂ levels and increasing Aβ₃₈ levels, while not affecting Aβ₄₀ levels. In yet another aspect of this embodiment, this method can increase the levels of other Aβ proteins smaller than Aβ₄₀, including Aβ₃₄, Aβ₃₇, Aβ₃₈, and Aβ₃₉.

In a seventh embodiment, the invention provides a method for treating neurodegenerative disorders while avoiding and/or reducing the side-effect associated with higher levels of the R-NSAID and NMDA antagonist. Side-effects associated with either of the active ingredients, the R-NSAID and NMDA antagonist are known to the skilled artisan. For example, the R-NSAID may be administered as the R-enantiomer substantial free of the S-enantiomer or as apart of a racemic mixture, but at levels (or by treatment regimes) which reduce the side-effect associated with the S-enantiomer. NMDA antagonists are known to have a variety of associated side-effects. According to the method of this embodiment, a disease treating or preventing effective amount of at least one R-NSAID and at least one NMDA antagonist is administered to an individual in need of such treatment, at levels (or by treatment regimes) that avoid the side-effects associated with these treatments. The individual in need of treatment can have a neurodegenerative disorder, a predisposition to a neurodegenerative disorder, and/or desire prophylaxis against neurodegenerative disorders. In one aspect of this embodiment, the effective amount of the at least one R-NSAID and at least one NMDA antagonist is capable of reducing at least one symptom of the neurodegenerative disorder. In another aspect, for individuals desiring prophylaxis against a neurodegenerative disorder, the effective amount of the at least one R-NSAID and at least one NMDA antagonist, is capable of preventing an increase (or increase in rate of increase) in at least one symptom of the neurodegenerative disorder. In one aspect of this method, the at least one NMDA antagonist is memantine. In another aspect of this method, the at least one NMDA antagonist is selected from the group consisting of memantine, adamantane, amantadine, an adamantane derivative, dextromethorphan, dextrorphan, dizocilpine, ibogaine, ketamine, remacemide, and phencyclidine. In one aspect of this method, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. In yet another aspect of this method, the NMDA antagonist is selected from the group consisting of memantine, adamantane, amantadine, an adamantane derivative, dextromethorphan, dextrorphan, dizocilpine, ibogaine, ketamine, remacemide, and phencyclidine, and the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. In still another aspect of this method, the R-NSAID is R-flurbiprofen. In another aspect of this method the R-NSAID is R-flurbiprofen and the NMDA antagonist is selected from the group consisting of memantine, adamantane, amantadine, an adamantane derivative, dextromethorphan, dextrorphan, dizocilpine, ibogaine, ketamine, remacemide, and phencyclidine. The method of the invention further provides for the treatment or prophylaxis of neurodegenerative disorders by administering an effective amount of R-flurbiprofen and memantine; R-flurbiprofen and adamantane; R-flurbiprofen and amantadine; R-flurbiprofen and an adamantane derivative; R-flurbiprofen and dextromethorphan; R-flurbiprofen and dextrorphan; R-flurbiprofen and dizocilpine; R-flurbiprofen and ibogaine; R-flurbiprofen and ketamine; R-flurbiprofen and ketamine; R-flurbiprofen and remacemide; or R-flurbiprofen and phenylcyclidine. In a preferred aspect of this method, the neurodegenerative disease is selected from the group consisting of Alzheimer's disease, dementia, and mild cognitive impairment. In another preferred embodiment, the invention provides a method for the treatment or prophylaxis of Alzheimer's disease through the administration of an effective amount of R-flurbiprofen and memantine; R-flurbiprofen and adamantane; R-flurbiprofen and an adamantane derivative; R-flurbiprofen and dextromethorphan; R-flurbiprofen and dextrorphan; R-flurbiprofen and dizocilpine; R-flurbiprofen and ibogaine; R-flurbiprofen and ketamine; R-flurbiprofen and ketamine; R-flurbiprofen and remacemide; or R-flurbiprofen and phenylcyclidine.

In an eighth embodiment, the invention provides compositions and a method for treating and/or preventing neurodegenerative disorders by administering, to an individual in need of such treatment, an effective amount of at least one R-NSAID, at least one NMDA antagonist such as memantine, adamantane, amantadine, an adamantane derivative, dextromethorphan, dextrorphan, dizocilpine, ibogaine, ketamine, remacemide, and phencyclidine, and at least one compound selected from the group consisting of secretase inhibitors, acetylcholine esterase inhibitors, GABA-A alpha 5 inverse agonists, and antioxidants. The combination can be administered simultaneously or separately.

In a ninth embodiment, the invention provides a method of lowering Aβ₄₂ levels to a greater extent than inhibiting COX-1, COX-2, or a combination thereof. In particular, the method of this embodiment involves administering to a patient, in need of treatment, an effective amount of at least one R-NSAID and at least one NMDA antagonist. According to one aspect of this embodiment, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. According to another aspect of this embodiment, the NMDA antagonist is selected from the group consisting of memantine, adamantane, amantadine, an adamantane derivative, dextromethorphan, dextrorphan, dizocilpine, ibogaine, ketamine, remacemide, and phencyclidine. In another aspect of this embodiment, the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac, and the NMDA antagonist is selected from the group consisting of memantine, adamantane, amantadine, an adamantane derivative, dextromethorphan, dextrorphan, dizocilpine, ibogaine, ketamine, remacemide, and phencyclidine. In a preferred aspect of this embodiment, the R-NSAID is R-flurbiprofen and the NMDA antagonist is selected from the group consisting of memantine, adamantane, amantadine, an adamantane derivative, dextromethorphan, dextrorphan, dizocilpine, ibogaine, ketamine, remacemide, and phencyclidine. The method of this embodiment involves the lowering of Aβ₄₂ levels while not substantial affecting the activity of COX-1, COX-2, or both COX-1, and COX-2. Thus, the amount that is administered is effective for lowering Aβ₄₂ levels and does not substantially inhibit COX-1, COX-2, or both COX-1 and COX-2. For example, the effective amount can be above the ED₅₀ (the dose therapeutically effective in 50% of the population) for Aβ₄₂ lowering, and below the ED₅₀ for COX inhibition. Another example is a sufficiently small amount of compound so that inhibition of at least one COX activity is negligible (suitable for chronic therapeutic or prophylactic use) and Aβ₄₂ levels are reduced. The method of this embodiment can be used to treat and/or prevent Alzheimer's disease. The method of this embodiment can also be used to treat and/or prevent MCI, dementia, and other neurodegenerative disorders.

In a tenth embodiment, the invention provides a composition comprising at least one NMDA antagonist and at least one Aβ₄₂ lowering agent. In one aspect of this embodiment, the at least one NMDA antagonist is selected from the group consisting of memantine, adamantane, amantadine, an adamantane derivative, dextromethorphan, dextrorphan, dizocilpine, ibogaine, ketamine, remacemide, and phencyclidine. In another aspect of this embodiment the at least one NMDA antagonist is memantine. In one aspect of this embodiment the Aβ₄₂ lowering agent is chosen from R-flurbiprofen, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. In yet another aspect of this embodiment the NMDA antagonist is selected from the group consisting of memantine, adamantane, amantadine, an adamantane derivative, dextromethorphan, dextrorphan, dizocilpine, ibogaine, ketamine, remacemide, and phencyclidine, and the Aβ₄₂ lowering agent is chosen from R-flurbiprofen, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. In still another aspect of this embodiment, the Aβ₄₂ lowering agent is R-flurbiprofen. The compositions of this embodiment can provide the two components together in a single dose with a pharmaceutically acceptable carrier.

In an eleventh embodiment, the invention provides a method for treating or preventing neurodegenerative disorders such as Alzheimer's disease. In particular, this method relates to treating or delaying the onset of neurodegenerative disorders by administering to an individual a therapeutically or prophylactically effective amount of at least one NMDA antagonist and at least one Aβ₄₂ lowering agent. This method may treat (or slow the onset of the progression of) the disease or disorder. This method may also be used to delay or slow the onset of the disease or disorder or signs or symptoms thereof.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

The invention generally relates to compositions and therapeutic treatments for neurodegenerative disorders, particularly Alzheimer's disease, MCI, Down's syndrome, and tauopathies (e.g., corticobasal degeneration, frontotemporal dementia with Parkinsonism linked to chromosome 17, and progressive supranuclear palsy, etc.). More specifically, the invention provides a composition for treating, delaying the onset, and preventing neurodegenerative disorders. One composition of the invention has at least one NMDA (N-methyl-D-aspartate) antagonist and at least one R-NSAID (non-steroidal anti-inflammatory) and at least one pharmaceutically acceptable carrier. Another composition of the invention comprises two compounds that are capable of interacting with CYP2C9, wherein at least one of the compounds is an Aβ₄₂ lowering agent. One method of the invention involves treating an individual in need of treatment (or prophylaxis) with a therapeutically (or prophylactically) effective amount of at least one NMDA antagonist and at least one R-NSAID. This method of the invention can involve co-administering the at least one NMDA antagonist and the at least one R-NSAID, or the at least one NMDA antagonist and the at least one R-NSAID can be administered to the same individual at different times and/or by different routes of administration. For example, the NMDA antagonist can be administered in the morning and the R-NSAID can be administered in the evening. Without wishing to be bound by any theory, it is believed that the combination of an R-NSAID and an NMDA antagonist is unexpectedly useful for treating Alzheimer's disease patients.

Another method of the invention involves treating an individual with a therapeutically or prophylactically effective amount of at least two compounds that are capable of interacting with CYP2C9, wherein at least one of the compounds is an Aβ₄₂ lowering agent. While not wishing to be bound by any theory, it is believed that when these compounds are administered to an individual, the compounds act synergistically in vivo to treat and/or prevent diseases or disorders characterized by increased levels of Aβ₄₂. For example, Alzheimer's disease, mild cognitive impairment (MCI), and/or other neurodegenerative diseases may be treated or have the onset delayed by the methods of the invention because increased Aβ₄₂ levels are associated with these diseases.

It is thought that by treating an individual with two CYP2C9 interacting compounds, wherein at least one of the compounds is an Aβ₄₂ lowering agent, the CYP2C9 enzyme will show a marked decrease in its ability to metabolize the Aβ₄₂ lowering compounds due to the synergistic properties of the composition. Therefore, the methods of the present invention lower Aβ₄₂ levels and thus treat or delay the onset of Alzheimer's disease, dementia, MCI, Down's syndrome, tauopathies (e.g., corticobasal degeneration, frontotemporal dementia with Parkinsonism linked to chromosome 17, and progressive supranuclear palsy, etc.), and other Aβ₄₂ related disorders.

It is believed that treating an individual with an Aβ₄₂-lowering NSAID (e.g. flurbiprofen, ibuprofen), or a derivative or analogue thereof, and fluvastatin or rosuvastatin, or a derivative or analogue thereof, will have the unexpected property of reducing the toxicity to the gastrointestinal system of the usual treatment of an individual with flurbiprofen. Although not wishing to be bound by any one theory, it is thought that the toxicity of the compound may be reduced because the Aβ₄₂ lowering effect of treating an individual with flurbiprofen can be achieved by administering a smaller amount of flurbiprofen. For example, smaller amounts of flurbiprofen may be administered to achieve the same Aβ₄₂ lowering effect because of the inhibitory effect of fluvastatin on CYP2C9 and/or because of the Aβ₄₂ lowering effect of fluvastatin. Therefore, the methods of the present invention effectively lower Aβ₄₂ levels with lower toxicity and thus treat or prevent Alzheimer's disease, dementia, MCI, Down's syndrome, tauopathies (e.g., corticobasal degeneration, frontotemporal dementia with Parkinsonism linked to chromosome 17, and progressive supranuclear palsy, etc.) and other Aβ₄₂ related disorders.

Accordingly, the invention provides a method for treating neurodegenerative disorders while avoiding and/or reducing the side-effects associated with higher levels of compounds that are capable of interacting with CYP2C9, wherein at least one of the compounds is an Aβ₄₂ lowering agent. In one embodiment, at least one of the compounds is a substrate of CYP2C9, such as diclofenac, ibuprofen, meloxicam, naproxen, piroxicam, suprofen, flurbiprofen, celicoxib, tolbutamide, glipizide, losartan, irbesartan, amitriptyline, fluoxetine, fluvastatin, phenyloin, rosiglitazone, tamoxifen, torsemide, S-warfarin, or naphthalene. In another embodiment, at least one of the compounds is a CYP2C9 inhibitor, such as amiodarone, atorvastatin, cerivastatin, fluconazole, fluvastatin, fluvoxamine, isoniazid, lovastatin, paroxetine, phenylbutazone, probenicid, sertraline, simvastatin, sulfamethoxazole, sulfaphenazole, sulphinpyrazone, teniposide, trimethoprim, zafirlukast, or rosuvastatin. In a specific example, at least one of the compounds is R-flurbiprofen or ibuprofen and at least one of the compounds is a statin, such as fluvastatin or rosuvastatin.

The invention provides a composition having at least one NMDA antagonist and at least one R-NSAID. The NMDA antagonists used in the invention can be any NMDA antagonist. Preferred NMDA antagonists are selected from the group consisting of memantine, adamantane, amantadine, an adamantane derivative, dextromethorphan, dextrorphan, dizocilpine, ibogaine, ketamine, remacemide, and phencyclidine. Preferred R-NSAIDs are selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. Preferably, the NMDA antagonist is selected from the group consisting of memantine, adamantane, amantadine, an adamantane derivative, dextromethorphan, dextrorphan, dizocilpine, ibogaine, ketamine, remacemide, and phencyclidine, and the R-NSAID is selected from the group consisting of R-flurbiprofen, R-ibuprofen, R-ketoprofen, R-ketorolac, R-naproxen, R-tiaprofenic acid, R-suprofen, R-carprofen, R-pirprofen, R-indoprofen, R-benoxaprofen, and R-etolodac. A preferred composition of the invention has R-flurbiprofen and an NMDA antagonist. Another preferred composition has R-flurbiprofen and an NMDA antagonist selected from the group consisting of memantine, adamantane, amantadine, an adamantane derivative, dextromethorphan, dextrorphan, dizocilpine, ibogaine, ketamine, remacemide, and phencyclidine. The invention further provides a composition having R-flurbiprofen and memantine; R-flurbiprofen and adamantane; R-flurbiprofen and amantadine; R-flurbiprofen and an adamantane derivative; R-flurbiprofen and dextromethorphan; R-flurbiprofen and dextrorphan; R-flurbiprofen and dizocilpine; R-flurbiprofen and ibogaine; R-flurbiprofen and ketamine; R-flurbiprofen and ketamine; R-flurbiprofen and remacemide; or R-flurbiprofen and phenylcyclidine. Without wishing to be bound by theory, it is believed the compositions of the invention are unexpectedly useful for treating neurodegenerative disorders and may exhibit a synergistic effect when used in combination for treating neurodegenerative disorders.

The invention also provides compositions comprising two compounds that are capable of interacting with CYP2C9, wherein at least one of the compounds is an Aβ₄₂ lowering agent. In one embodiment, at least one of the compounds is a substrate of CYP2C9. For example, the CYP2C9 substrate may be selected from the group consisting of diclofenac, ibuprofen, meloxicam, naproxen, piroxicam, suprofen, flurbiprofen, celicoxib, tolbutamide, glipizide, losartan, irbesartan, amitriptyline, fluoxetine, fluvastatin, phenyloin, rosiglitazone, tamoxifen, torsemide, S-warfarin, naphthalene. In a specific example the CYP2C9 substrate is an NSAID, preferably an R-NSAID such as R-flurbiprofen or R-ibuprofen.

In another embodiment, at least one of the compounds is a CYP2C9 inhibitor. For example, the CYP2C9 inhibitor may be selected from the group consisting of amiodarone, atorvastatin, cerivastatin, fluconazole, fluvastatin, fluvoxamine, isoniazid, lovastatin, paroxetine, phenylbutazone, probenicid, sertraline, simvastatin, sulfamethoxazole, sulfaphenazole, sulphinpyrazone, teniposide, trimethoprim, zafirlukast, rosuvastatin. In a specific example the CYP2C9 inhibitor is a statin, preferably fluvastatin or rosuvastatin.

In yet another embodiment, one of the compounds is a CYP2C9 substrate and one of the compounds is a CYP2C9 inhibitor. For example, one of the compounds may be a CYP2C9 substrate and one of the compounds may be a statin, preferably fluvastatin or rosuvastatin. In a specific example, one of the CYP2C9 inhibitors is amiodarone, atorvastatin, cerivastatin, fluconazole, fluvastatin, fluvoxamine, isoniazid, lovastatin, paroxetine, phenylbutazone, probenicid, sertraline, simvastatin, sulfamethoxazole, sulfaphenazole, sulphinpyrazone, teniposide, trimethoprim, zafirlukast, or rosuvastatin and one of the CYP2C9 substrates is diclofenac, ibuprofen, meloxicam, naproxen, piroxicam, suprofen, flurbiprofen, celicoxib, tolbutamide, glipizide, losartan, irbesartan, amitriptyline, fluoxetine, fluvastatin, phenyloin, rosiglitazone, tamoxifen, torsemide, S-warfarin, or napthalene. In yet another specific aspect of this embodiment one of the compounds is an NSAID, preferably an R-NSAID, and one of the compounds is amiodarone, atorvastatin, cerivastatin, fluconazole, fluvastatin, fluvoxamine, isoniazid, lovastatin, paroxetine, phenylbutazone, probenicid, sertraline, simvastatin, sulfamethoxazole, sulfaphenazole, sulphinpyrazone, teniposide, trimethoprim, zafirlukast, or rosuvastatin. In a preferred example, at least one of the compounds is R-flurbiprofen or ibuprofen and at least one of the compounds is a statin, preferably fluvastatin or rosuvastatin.

In still another embodiment, at least one of the interactors of CYP2C9 is a modified NSAID, NSAID derivative, or NSAID analogue, which can be prepared by a variety of methods known in the art. A typical way of producing a modified NSAID is an addition (e.g. adding alkyl, hydroxyl alkyl, phenyl, benzyl, or thienyl groups) to an indole. NSAIDs can also be modified by substituting functional groups (e.g. substituting an ester for an acid. In a preferred embodiment, the NSAID derivative or analogue of the present invention is a derivative or analogue of R-flurbiprofen. Specifically, the compounds of the present invention include analogues of flurbiprofen such as 2-(2′-fluro-4-biphenylyl) propionic acid and 2-(2,2′-difluroro-4-biphenylyl) propionic acid, both found in U.S. Pat. No. 3,755,427, which is incorporated herein by reference, and derivatives of flurbiprofen such as 4′-hydroxyflurbiprofen.

In another embodiment at least one of the interactors of CYP2C9 is a derivative or analogue of a statin. Specifically, one of the compounds may be a derivative or analogue of fluvastatin such as found in U.S. Pat. No. 4,739,073, which is incorporated herein by reference. Statins, and derivative or analogues thereof, also include compounds of the formula: 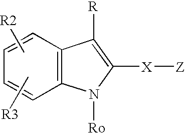
wherein one of R and Ro is 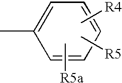
and the other is a primary or secondary C₁₋₆alklyle not containing an asymmetric carbon atom, C₃₋₆cycloalkyl or phenyl-(CH₂)ₘ—, wherein
R4 is hydrogen, C1-3alkyl, n-butyl, i-butyl, t-butyl, C1-3alkoxy, n-butoxy, i-butoxy, trifluoromethyl, fluro, chloro, phenoxy or benzyloxy,
R5 is hyrodgen, C1-3alkyl, C1-3alkoxy, trifluoro-methyl, fluoro, chloro, phenoxy or benzyloxy,
R5a is hydrogen, C1-2alkyl, C1-2alkyl, C1-2alkoxy, fluoro or chloro, and m is 1, 2 or 3, provided that both R5 and R5a are hydrogen when R4 is hydrogen, R5a is hydrogen when R5 is hydrogen, not more than one of R4 and R5 is trifluoromethyl, not more than one of R4 and R5 is phenoxy, and not more than one of of R4 and R5 is benzyloxy,
R2 is hydrogen, C1-3alkyl, n-butyl, i-butyl, t-butyl, C4-6alkoxy, n-butoxy, i-butoxy, trifluoromethyl, fluro, chloro, phenoxy or benzyloxy,
R3 is hydrogen, C1-3alkyl, n-butyl, i-butyl, t-butyl, C4-6alkoxy, n-butoxy, i-butoxy, trifluoromethyl, fluro, chloro, phenoxy or benzyloxy, provided that R3 is hydrogen when R2 is hydrogen, not more than one of R2 and R3 is trifluoromethyl, not more than one of R2 and R3 is phenoxy, and not more than one of R2 and R3 is benzyloxy,
X is —(CH2)n-, or —CH═CH—, wherein n is 0, 1, 2 or 3, and
Z is 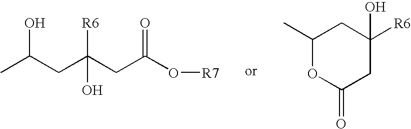
wherein
   R6 is hydrogen or C1-3alkyl, and
   R7 is hydrogen, R7b or M,
wherein
   R7b is a physiologically acceptable and hydrolysable ester group, and
   M is a pharmaceutically acceptable cation.

Statins, and analogues and derivatives thereof, also include the compositions disclosed in U.S. Pat. No. 5,260,4004, which is incorporated herein by reference. For example, statins, and analogues and derivatives thereof, may have the formula: 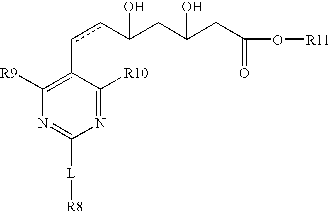
wherein:
R8 is lower alkyl, aryl, or aralkyl, each of which may have one or more substituents;
R9 and R10 each is independently hydrogen, lower alkyl, or aryl and each of the lower alkyl and aryl may have one or more substituents;
R11 is hydrogen, lower alkyl, or a cation capable of forming a non-toxic pharmaceutically acceptable or ester;
L is sulfur, oxygen, or sulfonyl, or imino which may have a substituent; and
the dotted line represents the presence or absence of a double bond, or the corresponding ring-closed lactone.

In another embodiment, at least one of the CYP2C9 interactors is nitrosated or nitrosylated (e.g. nitrosated or nitrosylated NSAIDs, or derivatives or analogues therof). Nitrosation refers to linking a nitrogen monoxide group (NO) to a compound. Nitrosylation refers to linking a nitrogen dioxide group (NO₂) to a compound. Nitrosated and/or nitrosylated NSAIDs and nitrosated and/or nitrosylated NSAID derivatives are known to release nitric oxide, which may increase the efficacy of clearing Aβ deposits in an individual. (See Jantzen et al., Journal of Neuroscience, 22:2246-2254 (2002)). Examples of nitrosated and/or nitrosylated NSAIDS are found in U.S. patent application Ser. No. 938,560, which is incorporated herein by reference. Specifically, one of the compounds is nitrosated and/or nitrosylated flurbiprofen or R-flurbiprofen.

In another embodiment, at least one of the CYP2C9 interactors has a sulfur-containing functional group containing a hydrocarbyl moiety covalently attached. Examples of NSAIDs attached to sulfur-containing functional groups are found in U.S. Pat. No. 6,355,666, which is incorporated herein by reference. In a specific example, the modified NSAID may have the structure:
A-D-Y—S(O)ₙ-M-Q
wherein:
A is an NSAID;
D is an optional linker/spacer;
Y and M are optionally present, and when present are independently —O— or —NT-, wherein T is H or an optionally substituted hydrocarbyl moiety;
n is 1 or 2; and
Q is H or an optionally substituted hydrocarbyl moiety.

In another embodiment, one of the CYP2C9 interacting compounds is capable of lowering Aβ₄₂ levels and increasing Aβ₃₈ levels, while not affecting Aβ₄₀ levels. The composition of this embodiment can also increase the levels of other Aβ proteins smaller than Aβ₄₀, including Aβ₃₄, Aβ₃₇, Aβ₃₈, and Aβ₃₉.

The compositions of the invention can provide the compounds together in a single dosage form or in separate dosage forms. The compositions of the invention can also provide the compounds with a pharmaceutically acceptable carrier.

The pathological hallmarks of Alzheimer's disease are most prevalent in the brain regions involved in higher cognitive functions. These features include a marked loss of neurons and synapses, numerous extracellular neuritic (senile) plaques and intracellular neurofibrillary tangles. The plaques are formed by a core of amyloid material surrounded by a halo of dystrophic neurites. The major component of the core is a peptide of 37 to 43 amino acids in length called the amyloid beta protein (Aβ), the major forms being Aβ₄₀ and Aβ₄₂. The tangles are formed by paired helical filaments, the major component of which is a hyperphosphorylated form of the microtubule-associated protein tau (τ). A large body of evidence suggests that the metabolism of APP and the generation of the Aβ peptide are central in AD pathogenesis. In fact, APP metabolism is regarded as the biochemical link between the pathology and genetics of AD. In addition, lowering Aβ₄₂ leads to clearance of tau pathology and treatment of tauopathies.

In one aspect, the invention provides a method of treating a neurodegenerative disorder, by identifying a patient in need of such treatment, and administering to the patient a therapeutically effective amount of a pharmaceutical composition having one or more R-NSAIDS (i.e., R-flurbiprofen) and one or more NMDA antagonists (i.e., memantine.) In another aspect, this method comprises administering a therapeutically effective amount of at least two compounds capable of interacting with CYP2C9, wherein at least one of said compounds is an Aβ₄₂ lowering agent, is administered to an individual who desires or is in need of such treatment. Administration of a compound of one or more R-NSAIDS (i.e., R-flurbiprofen) and one or more NMDA antagonists (i.e., memantine) for at least 4 weeks, preferably at least 4 months, and more desirably at least 8 months, can provide an improvement or lessening in decline of cognitive function as characterized by cognition tests, biochemical disease marker progression, and/or plaque pathology. Cognition tests are those which are capable of measuring cognitive decline in a patient or group of patients. Examples of such cognition tests include the ADAS-cog (Alzheimer's Disease Assessment Scale, cognitive subscale), NPI (Neuropsychiatric Inventory), ADCS-ADL (Alzheimer's Disease Cooperative Study-Activities of Daily Living), CIBIC-plus (Clinician Interview Based Impression of Change), and CDR sum of boxes (Clinical Dementia Rating). It is preferred that the lessening in decline in cognitive function is at least 25% as compared to individuals treated with placebo, more preferably at least 40%, and even more desirably at least 60%. For example, an individual treated with placebo having probable mild-to-moderate Alzheimer's disease is expected to score approximately 5.5 points worse on the ADAS-cog test after a specified period of time of treatment (e.g., 1 year) whereas an individual treated with the composition of this aspect of the invention for the same period of time will score approximately 2.2 points worse on the ADAS-cog scale with a 60% decrease in decline or 3.3 points worse with a 40% decrease in decline in cognitive function when treated with the composition for the same specified period of time. Desirably, the oral dose is provided in capsule or tablet form. The pharmaceutical composition for use in the invention is formulated with one or more pharmaceutically acceptable excipients, salts, or carriers. The pharmaceutical composition for use in the invention is delivered orally, preferably in a tablet or capsule dosage form.

In another aspect, the invention provides a method of treating mild-to-moderate Alzheimer's disease by identifying a patient having mild-to-moderate Alzheimer's disease and administering to the patient an Alzheimer's disease treating effective amount of one or more R-NSAIDS (i.e., R-flurbiprofen) and one or more NMDA antagonists (i.e., memantine.) In another aspect, this method comprises administering a therapeutically effective amount of at least two compounds capable of interacting with CYP2C9, wherein at least one of said compounds is an Aβ₄₂ lowering agent, is administered to an individual who desires or is in need of such treatment. One criterion indicating a likelihood of mild-to-moderate Alzheimer's disease is a score of about 15 to about 26, or about 19 to about 21, on the MMSE test. Another criteria indicating mild-to-moderate Alzheimer's disease is a decline in cognitive function. In specific embodiments, an individual who desires or is in need of treatment has an MMSE test score of from about 26 to about 19, inclusive. In additional embodiments, an individual who desires or is in need of treatment has an MMSE test score of from about 18 to about 10, inclusive. In a specific embodiment, said individual has an MMSE test score of about 26 to about 10, inclusive.

Oral administration of one or more R-NSAIDS (i.e., R-flurbiprofen) and one or more NMDA antagonists (i.e., memantine) according to this aspect of the invention, for at least 4 weeks, preferably at least 4 months, and more desirably at least 8 months, provides an improvement or lessening in decline of cognitive function as characterized by cognition tests, biochemical disease marker progression, and/or plaque pathology. Desirably, the dose is administered orally and is provided in capsule or tablet form. The method of this aspect of the invention involves identifying an individual likely to have mild-to-moderate Alzheimer's disease. An individual having probable mild-to-moderate Alzheimer's disease can be diagnosed by any method available to the ordinary artisan skilled in such diagnoses. For example, diagnosis can be according to DSM IV (TR) and/or meets NINCDS-ADRDA criteria for probable AD. According to this aspect of the invention, individuals with probable mild-to-moderate AD is administered preferably by an oral route, one or more R-NSAIDS (i.e., R-flurbiprofen) and one or more NMDA antagonists (i.e., memantine) for a period of time. Individuals undergoing such treatment are likely to see an improvement or lessening in decline of cognitive function, an improvement or lessening in decline in biochemical disease marker progression, and/or an improvement or lessening decline in plaque pathology. A lessening in decline in cognitive function can be assessed using test of cognitive function like the ADAS-cog. For example, an individual treated with placebo having probable mild-to-moderate Alzheimer's disease is expected to score approximately 5.5 points worse on the ADAS-cog test after a specified period of time of treatment (e.g., 1 year) whereas an individual treated with the composition of this aspect of the invention for the same period of time will score approximately 2.2 points worse on the ADAS-cog scale with a 60% decrease in decline or 3.3 points worse with a 40% decrease in decline in cognitive function when treated with the composition for the same specified period of time.

In another aspect, the invention provides a method of treating moderate-to-severe Alzheimer's disease by identifying a patient having moderate-to-severe Alzheimer's disease and administering to the patient an Alzheimer's disease treating effective amount of one or more R-NSAIDS (i.e., R-flurbiprofen) and one or more NMDA antagonists (i.e., memantine.) In another aspect, this method comprises administering a therapeutically effective amount of at least two compounds capable of interacting with CYP2C9, wherein at least one of said compounds is an Aβ₄₂ lowering agent, is administered to an individual who desires or is in need of such treatment. Oral administration of one or more R-NSAIDS (i.e., R-flurbiprofen) and one or more NMDA antagonists (i.e., memantine) according to this aspect the invention, for at least 4 weeks, preferably at least 4 months, and more desirably at least 8 months, provides an improvement or lessening in decline of cognitive function as characterized by cognition tests, biochemical disease marker progression, and/or plaque pathology. Desirably, the dose is administered orally and is provided in capsule or tablet form. The method of this aspect of the invention involves identifying an individual likely to have moderate-to-severe Alzheimer's disease. An individual having moderate-to-severe Alzheimer's disease can be diagnosed by any method available to the ordinary artisan skilled in such diagnoses. For example, diagnosis can be according to DSM IV (TR) and/or meets NINCDS-ADRDA criteria for probable AD. According to this aspect of the invention, individuals with probable moderate-to-severe AD is administered preferably by an oral route, one or more R-NSAIDS (i.e., R-flurbiprofen) and one or more NMDA antagonists (i.e., memantine) for a period of time. Individuals undergoing such treatment are likely to see an improvement or lessening in decline of cognitive function, an improvement or lessening in decline in biochemical disease marker progression, and/or an improvement or lessening decline in plaque pathology. A lessening in decline in cognitive function can be assessed using test of cognitive function like the ADAS-cog. For example, an individual treated with placebo having probable moderate-to-severe Alzheimer's disease is expected to score approximately 5.5 points worse on the ADAS-cog test after a specified period of time of treatment (e.g., 1 year) whereas an individual treated with the composition of this aspect of the invention for the same period of time will score approximately 2.2 points worse on the ADAS-cog scale with a 60% decrease in decline or 3.3 points worse with a 40% decrease in decline in cognitive function when treated with the composition for the same specified period of time.

An AD diagnosis can be made using any known method. Typically, AD is diagnosed using a combination of clinical and pathological assessments. For example, progression or severity of AD can be determined using Mini Mental State Examination (MMSE) as described by Mohs et al. Int Psychogeriatr 8:195-203 (1996); Alzheimer's Disease Assessment Scale-cognitive component (ADAS-cog) as described by Galasko et al. Alzheimer Dis Assoc Disord, 11 suppl 2:S33-9 (1997); the Alzheimer's Disease Cooperative Study Activities of Daily Living scale (ADCS-ADL) as described by McKhann et al. Neurology 34:939-944 (1984); and the NINCDS-ADRDA criteria as described by Folstein et al. J. Psychiatr. Res. 12:189-198 (1975). In addition, methods that allow for evaluating different regions of the brain and estimating plaque and tangle frequencies can be used. These methods are described by Braak et al. Acta Neuropathol 82:239-259 (1991); Khachaturian Arch. Neuro. 42:1097-1105 (1985); Mirra et al. (1991) Neurology 41:479-486; and Mirra et al. Arch Pathol Lab Med 117:132-144 (1993).

In a preferred embodiment, the invention provides methods for lowering or preventing an increase in Aβ₄₂ levels in an individual in need of such treatment. It is believed that by lowering the amounts of Aβ₄₂ in an individual by administering an Aβ₄₂ lowering effective amount of an R-NSAID and an NMDA antagonist, as described herein, that Alzheimer's disease, dementia, and mild cognitive impairment can be treated or prevented. Generally, the method relates to the idea that administering, to an individual, an effective amount of at least one R-NSAID and at least one NMDA antagonist can lower Aβ₄₂ levels. Thus, diseases characterized by increased levels of Aβ₄₂, can be treated or prevented with the methods of this embodiment which are designed to lower Aβ₄₂ or prevent an increase in Aβ₄₂.

While not wishing to be bound by theory, it is believed that administration of at least one R-NSAID, e.g., R-flurbiprofen, and at least one NMDA antagonist, e.g., memantine, adamantane, amantadine, an adamantane derivative, dextromethorphan, dextrorphan, dizocilpine, ibogaine, ketamine, remacemide, and phencyclidine, may act in vivo, synergistically to treat and/or prevent Alzheimer's disease, dementia, and/or MCI. It is thought that by lowering the amount of Aβ₄₂ that is present or would be present in the absence of such treatment by treating with R-flurbiprofen and memantine, an unexpectedly useful benefit for treating mild-to-moderate and moderate to severe Alzheimer's disease may be achieved. Amyloid β polypeptides are derived from amyloid precursor proteins (APPs). A variety of amyloid β polypeptides are known including Aβ₃₄, Aβ₃₇, Aβ₃₈, Aβ₃₉, and Aβ₄₀. Increased Aβ₄₂ levels are associated with Alzheimer's disease, dementia, MCI. Thus, by lowering the amounts of Aβ₄₂, a treatment is provided for combating Alzheimer's disease and/or MCI.

Aβ₄₂ lowering agents for use in the invention can be a known Aβ₄₂ lowering agents such as R-flurbiprofen, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. Examples of Aβ₄₂ lowering agents for use in the combination formulations and treatments of the invention are given in, e.g., WO 01/78721, WO 2004/073705, WO 2004/064771, and WO 2004/074232 (each of which is herein incorporated by reference).

Aβ₄₂ lowering agents include, but are not limited to those having the following Formulae: 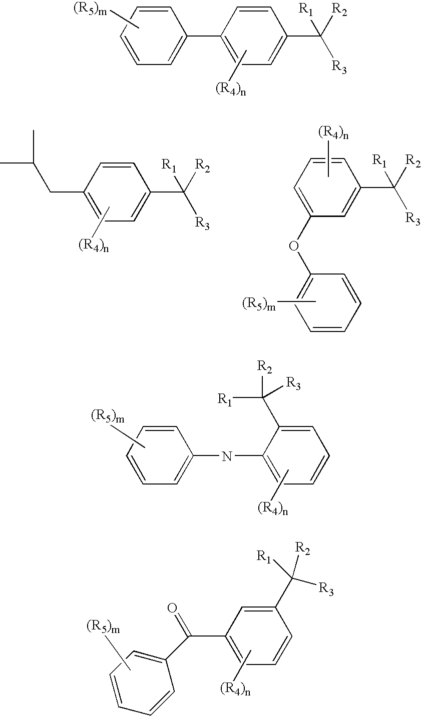
wherein R₁ is chosen from —CH₃, —CH₂CH₃, —CH₂CH₂CH₃, and —CH₂CH₂CH₂CH₃ (or can be taken together with R₂ to give a cyclopropyl ring, a cyclobutyl ring, a cyclopentyl ring, or a cyclohexyl ring);
R₂ is chosen from —CH₃, —CH₂CH₃, —CH₂CH₂CH₃, and —CH₂CH₂CH₂CH₃, (or can be taken together with R₂ to give a cyclopropyl ring, a cyclobutyl ring, a cyclopentyl ring, or a cyclohexyl ring);
R₃ is chosen from —COOH, —COOR₆, —CONH₂, —CONHR₆, —CONR₆R₇, —CONHSO₂R₆, tetrazolyl, and a —COOH bioisostere;
R₄ is chosen from Cl, —F, —Br, —I, —CF₃, —OCF₃, —SCF₃, —OCH₃, —OCH₂CH₃, —CN, —CH═CH₂, —CH₂OH, and —NO₂;
R₅ is chosen from —Cl, —F, —Br, —I, —CF₃, —OCF₃, —SCF₃, —OCH₃, —OCH₂CH₃, —CN, —CH═CH₂, —CH₂OH, and —NO₂;
R₆ is chosen from —CH₃, —CH₂CH₃, —CH₂CH₂CH₃, and —CH₂CH₂CH₂CH₃.

R₇ is chosen from —CH₃, —CH₂CH₃, —CH₂CH₂CH₃, and —CH₂CH₂CH₂CH₃.
M is an integer chosen from 0, 1, 2, and 3.
N is an integer chosen from 0, 1, 2, and 3.

Examples of compounds for use in the invention include those as shown above (and below), including enantiomers, diastereomers, racemates, and pharmaceutically acceptable salts thereof. The compounds described in this invention disclosure can be made by an ordinary artisan skilled in the art of organic chemistry synthesis.

Additional Aβ₄₂ lowering agents include, but are not limited to the following:

2-methyl-2 (2-fluoro-4′-trifluoromethylbiphen-4-yl) propionic acid; 2-methyl-2 (2-fluoro-4′-cyclohexyl biphen-4-yl) propionic acid; 1-(2-fluoro-4′-trifluoromethylbiphenyl-4-yl) cyclopropanecarboxylic acid; 1-(4′-cyclohexyl-2-fluorobiphenyl-4-yl) cyclopropanecarboxylic acid; 1-(4′-benzyloxy-2-fluorobiphenyl-4-yl) cyclopropanecarboxylic acid; 1-(2-fluoro-4′-isopropyloxybiphenyl-4-yl) cyclopropanecarboxylic acid; 1-(2-fluoro-3′-trifluoromethoxybiphenyl-4-yl) cyclopropanecarboxylic acid; 1-(2-fluoro-4′-trifluoromethoxybiphenyl-4-yl) cyclopropanecarboxylic acid; 1-(2-fluoro-3′-trifluoromethylbiphenyl-4-yl) cyclopropanecarboxylic acid; 1-(4′-cyclopentyl-2-fluorobiphenyl-4-yl) cyclopropanecarboxylic acid; 1-(4′-cycloheptyl-2-fluorobiphenyl-4-yl) cyclopropanecarboxylic acid; 1-(2′-cyclohexyl-2-fluorobiphenyl-4-yl) cyclopropanecarboxylic acid; 1-(2-fluoro-4′-hydroxybiphenyl-4-yl) cyclopropanecarboxylic acid; 1-[2-fluoro-4′-(tetrahydropyran-4-yloxy) biphenyl-4-yl]-cyclopropane-carboxylic acid; 1-(2,3′,4′-trifluorobiphenyl-4-yl) cyclopropanecarboxylic acid; 1-(3′,4′-dichloro-2-fluorobiphenyl-4-yl) cyclopropanecarboxylic acid; 1-(3′,5′-dichloro-2-fluorobiphenyl-4-yl) cyclopropanecarboxylic acid 1-(3′-chloro-2,4′-difluorobiphenyl-4-yl) cyclopropanecarboxylic acid; 1-(4-benzo[b]thiophen-3-yl-3-fluorophenyl) cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′:4′,1″] tert-phenyl-4″-yl)-cyclopropanecarboxylic acid; 1-[4′-(4,4-dimethylcyclohexyl)-2-fluoro [1,1′-biphenyl]-4-yl]-cyclopropane-carboxylic acid; 1-[2-fluoro-4′-[[4-(trifluoromethyl) benzoyl] ammino] [1,1′-biphenyl]-4-yl]-cyclopropanecarboxylic acid; 1-[2-fluoro-4′-[[4-(trifluoromethyl) cyclohexyl] oxy] [1,1′-biphenyl]-4-yl]-cyclopropanecarboxylic acid; 1-[2-fluoro-4′-[(3, 3,5,5-tetramethylcyclohexyl) oxy] [1,1′-biphenyl]-4-yl]-cyclopropanecarboxylic acid; 1-[4′-[(4,4-dimethylcyclohexyl) oxy]-2-fluoro [1,1′-biphenyl]-4-yl]-cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy [1,1′:4′,1″-tert-phenyl]-4-yl)-cyclopropane-carboxylic acid; 1-(2,2′-difluoro-4″-hydroxy [1,1′:4′,1″-tert-phenyl]-4-yl)-cyclopropane-carboxylic acid; 2-(2-fluoro-3′,5′-bis (chloro) biphen-4-yl) propionic acid amide; 2-(2-fluoro-4′-trifluoromethylbiphen-4-yl) propionic acid; 2-(2-fluoro-3′-trifluoromethylbiphen-4-yl) propionic acid; 2-(2-fluoro-3′,5′-bis (trifluoromethyl) biphen-4-yl) propionic acid; 2-(4′-cyclohexyl-2-fluorobiphen-4-yl) propionic acid; 2-(2-Fluoro-1,1′-biphenyl-4-yl)-2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole.

Aβ₄₂ lowering agents can be identified by a number of methods. To identify Aβ₄₂ lowering agents that reduce APP processing, a biological composition having an APP processing activity (i.e. an activity that processes APP into various Aβ forms, one of which is Aβ₄₂), is incubated with APP under conditions in which APP processing occurs. To identify Aβ₄₂ lowering agents that increase Aβ₄₂ catabolism, a biological composition having Aβ₄₂ catabolic activity is incubated with Aβ₄₂ under conditions in which Aβ₄₂ catabolism occurs. Depending on the nature of the biological composition, the APP or Aβ₄₂ substrate can be added to the biological composition, or, each or both can be a component of the biological composition. APP processing or Aβ₄₂ catabolism is allowed to take place in the presence or absence of the candidate Aβ₄₂ lowering agent. The level of Aβ₄₂ generated from APP processing or the level of Aβ₄₂ remaining after the catabolic reaction, in the presence and absence of the candidate Aβ₄₂ lowering agent, is determined and compared. Aβ₄₂ lowering agents useful for treating AD are those that reduce the level of Aβ₄₂ either by reducing APP processing into Aβ₄₂ or by enhancing Aβ₄₂ catabolism and increasing Aβ₃₈ production. The biological composition having an APP processing and/or catabolic activity can be a cell-free biological sample. For example, a cell-free biological sample can be a purified or partially purified enzyme preparation; it also can be a cell lysate generated from cells able to process APP into Aβ₄₂ or from cells able to catabolize Aβ₄₂. Cell lysates can be prepared using known methods such as, for example, sonication or detergent-based lysis. In the case of an enzyme preparation or cell lysate, APP can be added to the biological composition having the APP processing activity, or Aβ₄₂ can be added to the biological composition having Aβ₄₂ catabolic activity.

In addition, the biological composition can be any mammalian cell that has an APP processing activity as well as a nucleic acid vector encoding APP. Alternatively, the biological composition can be any mammalian cell that has Aβ catabolic activity as well as a nucleic acid vector or a viral nucleic acid-based vector containing a gene that encodes Aβ₄₂. The vector typically is an autonomously replicating molecule, a molecule that does not replicate but is transiently transfected into the mammalian cell, or a vector that is integrated into the genome of the cell. Typically, the mammalian cell is any cell that can be used for heterologous expression of the vector-encoded APP or Aβ₄₂ in tissue culture. For example, the mammalian cell can be a Chinese hamster ovary (CHO) cell, a fibroblast cell, or a human neuroglioma cell. The mammalian cell also can be one that naturally produces APP and processes it into Aβ₄₂, or one that naturally produces and catabolizes Aβ₄₂.

Further, the biological composition can be an animal such as a transgenic mouse that is engineered to over-express a form of APP that then is processed into Aβ₄₂. Alternatively, the animal can be a transgenic mouse that is engineered to over-express Aβ₄₂. Animals can be, for example, rodents such as mice, rats, hamsters, and gerbils. Animals also can be rabbits, dogs, cats, pigs, and non-human primates, for example, monkeys.

To perform an in vitro cell-free assay, a cell-free biological sample having an activity that can process APP into Aβ₄₂ is incubated with the substrate APP under conditions in which APP is processed into various Aβ forms including Aβ₄₂ (see Mclendon et al. (2000) FASEB 14:2383-2386). Alternatively, a cell-free biological sample having an activity that can catabolize Aβ₄₂ is incubated with the substrate Aβ₄₂ under conditions in which Aβ₄₂ is catabolized. To determine whether a candidate Aβ₄₂ lowering agent has an effect on the processing of APP into Aβ₄₂ or the catabolism of Aβ₄₂, two reactions are compared. In one reaction, the candidate Aβ₄₂ lowering agent is included in the processing or catabolic reaction, while in a second reaction, the candidate Aβ₄₂ lowering agent is not included in the processing or catabolic reaction. Levels of the different Aβ forms produced in the reaction containing the candidate Aβ₄₂ lowering agent are compared with levels of the different Aβ forms produced in the reaction that does not contain the candidate Aβ₄₂ lowering agent.

The different Aβ forms can be detected using any standard antibody based assays such as, for example, immunoprecipitation, western hybridization, and sandwich enzyme-linked immunosorbent assays (ELISA). Different Aβ forms also can be detected by mass spectrometry; see, for example, Wang et al. (1996) J Biol Chem 271:31894-902. Levels of Aβ species can be quantified using known methods. For example, internal standards can be used as well as calibration curves generated by performing the assay with known amounts of standards.

In vitro cell-based assays can be used determine whether a candidate Aβ₄₂ lowering agent has an effect on the processing of APP into Aβ₄₂ or an effect on catabolism of Aβ₄₂. Typically, cell cultures are treated with a candidate Aβ₄₂ lowering agent. Then the level of Aβ₄₂ in cultures treated with a candidate Aβ₄₂ lowering agent is compared with the level of Aβ₄₂ in untreated cultures. For example, mammalian cells expressing APP are incubated under conditions that allow for APP expression and processing as well as Aβ₄₂ secretion into the cell supernatant. The level of Aβ₄₂ in this culture is compared with the level of Aβ₄₂ in a similarly incubated culture that has been treated with the candidate Aβ₄₂ lowering agent. Alternatively, mammalian cells expressing Aβ₄₂ are incubated under conditions that allow for Aβ₄₂ catabolism. The level of Aβ₄₂ in this culture is compared with the level of Aβ₄₂ in a similar culture that has been treated with the candidate Aβ₄₂ lowering agent.

In vivo animal studies also can be used to identify Aβ₄₂ lowering agents useful for treating AD. Typically, animals are treated with a candidate Aβ₄₂ lowering agent and the levels of Aβ₄₂ in plasma, CSF, and/or brain are compared between treated animals and those untreated. The candidate Aβ₄₂ lowering agent can be administered to animals in various ways. For example, the candidate Aβ₄₂ lowering agent can be dissolved in a suitable vehicle and administered directly using a medicine dropper or by injection. The candidate Aβ₄₂ lowering agent also can be administered as a component of drinking water or feed. Levels of Aβ in plasma, cerebral spinal fluid (CSF), and brain are determined using known methods. For example, levels of Aβ₄₂ can be determined using sandwich ELISA or mass spectrometry in combination with internal standards or a calibration curve. Plasma and CSF can be obtained from an animal using standard methods. For example, plasma can be obtained from blood by centrifugation, CSF can be isolated using standard methods, and brain tissue can be obtained from sacrificed animals.

When present in an in vitro or in vivo APP processing or Aβ₄₂ catabolic reaction, Aβ₄₂ lowering agents reduce the level of Aβ₄₂ generated by APP processing or remaining following Aβ catabolism. For example, in an in vitro cell-free assay, the level of Aβ₄₂ is reduced due to either a reduction of APP processing or an increase in Aβ₄₂ catabolism in the presence the Aβ₄₂ lowering agent. In an in vitro cell culture study, a reduction in the level of Aβ₄₂ secreted into the supernatant results from the effect of the Aβ₄₂ lowering agent on either a reduction in processing of APP into Aβ₄₂ or an increased catabolism of Aβ₄₂. Similarly, in animal studies, a reduction in the level of Aβ₄₂ that can be detected in plasma, CSF, or brain is attributed to the effect of the Aβ₄₂ lowering agent on either a reduction in the processing of APP into Aβ₄₂ or an increase in the catabolism of Aβ₄₂.

The level of Aβ₄₂ can be reduced by a detectable amount. For example, treatment with an Aβ₄₂ lowering agent leads to at least about 0.5, 1, 3, 5, 7, 15, 20, 40, 50, or more than about 50% reduction in the level of Aβ₄₂ generated by APP processing or remaining following Aβ₄₂ catabolism when compared with that in the absence of the Aβ₄₂ lowering agent. Preferably, treatment with the Aβ₄₂ lowering agent leads to at least a 20% reduction in the level of Aβ₄₂ generated when compared to that in the absence of Aβ₄₂ lowering agent. More preferably, treatment with an Aβ₄₂ lowering agent leads to at least a 40% reduction the level of Aβ₄₂ when compared to that in the absence of an Aβ₄₂ lowering agent.

The invention also provides a composition having at least one NMDA antagonist and at least one Aβ₄₂ lowering agent. In one aspect of this embodiment, the at least one NMDA antagonist is selected from the group consisting of memantine, adamantane, amantadine, an adamantane derivative, dextromethorphan, dextrorphan, dizocilpine, ibogaine, ketamine, remacemide, and phencyclidine. In another aspect of this embodiment the at least one NMDA antagonist is memantine. In one aspect of this embodiment the Aβ₄₂ lowering agent is chosen from R-flurbiprofen, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. In yet another aspect of this embodiment the NMDA antagonist is selected from the group consisting of memantine, adamantane, amantadine, an adamantane derivative, dextromethorphan, dextrorphan, dizocilpine, ibogaine, ketamine, remacemide, and phencyclidine, and the Aβ₄₂ lowering agent is chosen from R-flurbiprofen, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. In still another aspect of this embodiment, the Aβ₄₂ lowering agent is R-flurbiprofen. In another aspect, the Aβ₄₂ lowering agent is R-flurbiprofen and the NMDA antagonist is selected from the group consisting of memantine, adamantane, amantadine, an adamantane derivative, dextromethorphan, dextrorphan, dizocilpine, ibogaine, ketamine, remacemide, and phencyclidine. The invention further provides compositions having R-flurbiprofen and memantine; R-flurbiprofen and adamantane; R-flurbiprofen and amantadine; R-flurbiprofen and an adamantane derivative; R-flurbiprofen and dextromethorphan; R-flurbiprofen and dextrorphan; R-flurbiprofen and dizocilpine; R-flurbiprofen and ibogaine; R-flurbiprofen and ketamine; R-flurbiprofen and remacemide; and R-flurbiprofen and phenylcyclidine. The compositions of this embodiment can provide the two components together in a single dose with a pharmaceutically acceptable carrier.

In another embodiment, the invention provides a method for treating or preventing neurodegenerative disorders such as Alzheimer's disease. In particular, this method relates to treating or delaying the onset of neurodegenerative disorders by administering to an individual a therapeutically or prophylactically effective amount of at least one NMDA antagonist and at least one Aβ₄₂ lowering agent.

For example, the invention provides a method of treating a neurodegenerative disorder, by identifying a patient in need of such treatment, and administering to the patient a therapeutically effective amount of one or more Aβ₄₂ lowering agents (i.e., R-flurbiprofen) and one or more NMDA antagonists (i.e., memantine.) Administration of one or more Aβ₄₂ lowering agents (i.e., R-flurbiprofen) and one or more NMDA antagonists (i.e., memantine) for at least 4 weeks, preferably at least 4 months, and more desirably at least 8 months, can provide an improvement or lessening in decline of cognitive function as characterized by cognition tests, biochemical disease marker progression, and/or plaque pathology. Desirably, the one or more Aβ₄₂ lowering agents and one or more NMDA antagonists are delivered in a pharmaceutical composition that is formulated with one or more pharmaceutically acceptable excipients, salts, or carriers. The pharmaceutical composition for use in the invention may be delivered orally, preferably in a tablet or capsule dosage form.

In another example, the invention provides a method of treating mild-to-moderate Alzheimer's disease by identifying a patient having mild-to-moderate Alzheimer's disease and administering to the patient an Alzheimer's disease treating effective amount of one or more Aβ₄₂ lowering agents (i.e., R-flurbiprofen) and one or more NMDA antagonists (i.e., memantine.) Oral administration of one or more Aβ₄₂ lowering agents (i.e., R-flurbiprofen) and one or more NMDA antagonists (i.e., memantine) according to this aspect the invention, for at least 4 weeks, preferably at least 4 months, and more desirably at least 8 months, provides an improvement or lessening in decline of cognitive function as characterized by cognition tests, biochemical disease marker progression, and/or plaque pathology.

In another aspect, the invention provides a method of treating moderate-to-severe Alzheimer's disease by identifying a patient having moderate-to-severe Alzheimer's disease and administering to the patient an Alzheimer's disease treating effective amount of one or more Aβ₄₂ lowering agents (i.e., R-flurbiprofen) and one or more NMDA antagonists (i.e., memantine.) Oral administration of one or more Aβ₄₂ lowering agents (i.e., R-flurbiprofen) and one or more NMDA antagonists (i.e., memantine) according to this aspect the invention, for at least 4 weeks, preferably at least 4 months, and more desirably at least 8 months, provides an improvement or lessening in decline of cognitive function as characterized by cognition tests, biochemical disease marker progression, and/or plaque pathology. Desirably, the dose is administered orally and is provided in capsule or tablet form. The method of this aspect of the invention involves identifying an individual likely to have moderate-to-severe Alzheimer's disease. An individual having moderate-to-severe Alzheimer's disease can be diagnosed by any method available to the ordinary artisan skilled in such diagnoses.

The invention also provides a method of treating neurodegenerative disorders (i.e. MCI and/or Alzheimer's disease) by identifying a patient having MCI or mild-to-moderate Alzheimer's disease and administering to the patient an amount of one or more Aβ₄₂ lowering agents (i.e., R-flurbiprofen) effective in treating MCI or mild-to moderate Alzheimer's disease until the patient reaches a moderate-to-severe stage of Alzheimer's disease and then administering to the patient an effective amount of one or more NMDA antagonists (i.e., memantine.) In one aspect of this embodiment, the at least one NMDA antagonist is selected from the group consisting of memantine, adamantane, amantadine, an adamantane derivative, dextromethorphan, dextrorphan, dizocilpine, ibogaine, ketamine, remacemide, and phencyclidine. In another aspect of this embodiment the at least one NMDA antagonist is memantine. In one aspect of this embodiment the Aβ₄₂ lowering agent is chosen from R-flurbiprofen, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. In yet another aspect of this embodiment the NMDA antagonist is selected from the group consisting of memantine, adamantane, amantadine, an adamantane derivative, dextromethorphan, dextrorphan, dizocilpine, ibogaine, ketamine, remacemide, and phencyclidine, and the Aβ₄₂ lowering agent is chosen from R-flurbiprofen, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. In still another aspect of this embodiment, the Aβ₄₂ lowering agent is R-flurbiprofen. In another aspect, the Aβ₄₂ lowering agent is R-flurbiprofen and the NMDA antagonist is selected from the group consisting of memantine, adamantane, amantadine, an adamantane derivative, dextromethorphan, dextrorphan, dizocilpine, ibogaine, ketamine, remacemide, and phencyclidine.

According to a preferred embodiment, the invention provides a method of lowering Aβ₄₂ levels to a greater extent than inhibiting COX-1, COX-2, or a combination thereof. In a preferred embodiment, the method comprises administering, to a patient in need of treatment, an effective amount of a R-NSAID, e.g., R-flurbiprofen, and at least one NMDA antagonist such as memantine, adamantane, amantadine, an adamantane derivative, dextromethorphan, dextrorphan, dizocilpine, ibogaine, ketamine, remacemide, and phencyclidine, wherein the effective amount of composition is capable of lowering Aβ₄₂, while not substantially affecting or inhibiting the activity of at least one isoform of COX. In another embodiment, the method comprises administering, to a patient in need of treatment, an effective amount of at least two CYP2C9 interacting compounds wherein at least one of said compounds is capable of lowering Aβ₄₂, while not substantially affecting or inhibiting the activity of COX-1, COX-2, or COX-3. Thus, the method of this embodiment involves the lowering of Aβ₄₂ levels while not substantially inhibiting the activity of COX-1, COX-2, or both COX-1 and COX-2. The method of this embodiment can be used to treat and/or prevent Alzheimer's disease, MCI, dementia, and/or other neurodegenerative disorders. In one aspect of this embodiment, the effective amount of the at least one R-NSAID, e.g., R-flurbiprofen and at least one NMDA antagonist, such as memantine, adamantane, amantadine, an adamantane derivative, dextromethorphan, dextrorphan, dizocilpine, ibogaine, ketamine, remacemide, and phencyclidine, reduces Aβ₄₂ levels or production of Aβ₄₂ by at least 1, 2, 5, 10, 15, 20, 25, 30, 40, or 50 or more percent while inhibiting COX-1, COX-2, or both COX-1 and COX-2 by less than 1, 2, 5, 10, 15, 20, 25, 30, 40, 50, 60, 70, 80, or 90 percent. In a preferred aspect of this embodiment, the effective amount of the R-NSAID, e.g., R-flurbiprofen, and at least one NMDA antagonist, such as memantine, adamantane, amantadine, an adamantane derivative, dextromethorphan, dextrorphan, dizocilpine, ibogaine, ketamine, remacemide, and phencyclidine, lowers Aβ₄₂ by at least 5 percent while not substantially inhibiting COX-1, COX-2, or both COX-1 and COX-2 activity or levels. In another preferred aspect of this embodiment, the effective amount of the R-NSAID, e.g., R-flurbiprofen, and at least one NMDA antagonist such as memantine, adamantane, amantadine, an adamantane derivative, dextromethorphan, dextrorphan, dizocilpine, ibogaine, ketamine, remacemide, and phencyclidine, that is administered to an individual is such that it lowers Aβ₄₂ levels, and does not inhibit COX activity to a significant extent, e.g., the amount administered is below the in vivo IC₅₀ value for COX-1, COX-2 or both COX-1 and COX-2 and above the in vivo IC₅₀ value for Aβ₄₂ lowering activity. As used in this context, IC₅₀ refers to the concentration of compound or composition sufficient to inhibit COX activity by 50% (COX-1, COX-2, or both COX-1 and COX-2) or reduce Aβ₄₂ levels (or rates of production) by 50%. An “effective amount” according to this preferred aspect of this embodiment, can also be viewed in terms of ED₅₀ parameters, binding constants, dissociation constants, and other pharmacological parameters, e.g., the amount administered is below the ED₅₀ value for COX-1, COX-2 or both COX-1 and COX-2 and above the ED₅₀ value for Aβ₄₂. It is noted that the effective amount of the compound does not necessarily have to be above an IC₅₀ or ED₅₀ for Aβ₄₂ lowering and below the IC₅₀ or ED₅₀ for COX inhibition. That is, the “effective amount” can be at some intermediate value such that Aβ₄₂ levels (or rates of production) are lowered to a greater extent than inhibition of COX-1, COX-2 or both COX-1 and COX-2. In one aspect, the method of this embodiment is thought to avoid the liability of adverse side effects associated with COX-1 and COX-2 inhibitors.

In another embodiment, the invention provides a method of lowering Aβ₄₂ levels and increasing Aβ₃₈ levels, while not affecting Aβ₄₀ levels. The method of this embodiment comprises administering, to an individual in need of such treatment, an effective amount of an R-NSAID, e.g., R-flurbiprofen, and at least one NMDA antagonist such as memantine, adamantane, amantadine, an adamantane derivative, dextromethorphan, dextrorphan, dizocilpine, ibogaine, ketamine, remacemide, and phencyclidine. The method according to this embodiment is useful for preventing and treating Alzheimer's disease. It is also contemplated that the method of this embodiment is useful for treating and preventing other disorders such as MCI, dementia, other neurodegenerative disorders. The Aβ₄₂ lowering method of this embodiment can also increase the levels of other Aβ proteins smaller than Aβ₄₀, including Aβ₃₄, Aβ₃₇, Aβ₃₈, and Aβ₃₉.

In another embodiment, the invention provides a method of lowering Aβ₄₂ levels and increasing Aβ₃₈ levels, while not affecting Aβ₄₀ levels. The method of this embodiment comprises administering, to an individual in need of such treatment, an effective amount of an effective amount of at least at least two compounds that are capable of interacting with CYP2C9, wherein at least one of the compounds is an Aβ₄₂ lowering agent. In one embodiment, at least one of the compounds is a substrate of CYP2C9. In another embodiment, at least one of the compounds is a CYP2C9 inhibitor. In a specific example, at least one of the compounds is R-flurbiprofen or ibuprofen and at least one of the compounds is a statin, preferably fluvastatin or rosuvastatin. The method according to this embodiment is useful for preventing and/or treating Alzheimer's disease. It is also contemplated that the method of this embodiment is useful for treating and preventing other disorders such as MCI, dementia, other neurodegenerative disorders. The Aβ₄₂ lowering method of this embodiment can also increase the levels of other Aβ proteins smaller than Aβ₄₀, including Aβ₃₄, Aβ₃₇, Aβ₃₈, and Aβ₃₉.

In another embodiment, the invention relates to a method of preventing Alzheimer's disease. According to this embodiment, a method for preventing Alzheimer's disease is provided which comprises administering, to an individual in need of such treatment, an effective amount of an R-NSAID, e.g., R-flurbiprofen, and at least one NMDA antagonist such as memantine, adamantane, amantadine, an adamantane derivative, dextromethorphan, dextrorphan, dizocilpine, ibogaine, ketamine, remacemide, and phencyclidine. The method of this embodiment is useful for preventing the symptoms of Alzheimer's disease, the onset of Alzheimer's disease, and/or the progression of the disease.

2.0. Definitions

As used herein, the term “preventing an increase in a symptom” refers both not allowing a symptom to increase or worsen, as well as reducing the rate of increase in the symptom. For example, a symptom can be measured as the amount of particular disease marker, i.e., a protein. Preventing an increase, according to the definition provided herein, means that the amount of the protein does not increase or that the rate at which it increases is reduced.

As used herein, the term “treating Alzheimer's disease” refers to a slowing of or a reversal of the progress of the disease. Treating Alzheimer's disease includes reducing the symptoms of the disease.

As used herein, the term “preventing Alzheimer's disease” refers to a slowing of of the onset of the disease or the symptoms thereof. Preventing Alzheimer's disease can include stopping the onset of the disease or symptoms thereof.

As used herein, the term “Aβ₄₂ lowering” refers the capability to reduce the amount of Aβ₄₂ present and/or being produced. Levels of Aβ₄₂ can be determined with an ELISA assay configured to detect Aβ₄₂. Methods of determining Aβ₄₂ levels are described in the examples and references cited therein. The term “Aβ₄₂ lowering effective amount” refers to an amount which reduces the amount of detectable Aβ₄₂ in cerebrospinal fluids in humans.

As used herein, the term “biometabolite” refers to a compound used according to the methods of the invention that is metabolized in vivo after administration to an individual.

As used herein, the term “biocleavable ester” refers to an ester derivative of a compound used in the invention that is cleavable in vivo to yield a the active form of the compound, a more active form of the compound, or a form of the compound that can be metabolized to yield an active compound.

As used herein, the term “NMDA antagonist” refers to class of pharmaceuticals known to interact with the NMDA receptor.

NMDA antagonists include, but are not limited to, adamantane derivative compounds such as 1-amino adamantane, 1-amino-3-phenyl adamantane, 1-amino-methyl-adamantane, 1-amino-3,5-dimethyl adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropyl-amino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, 1-amino-3-butyl-5-cyclohexyl adamantane, their N-methyl, N,N-dimethyl, N-ethyl, N-propyl derivatives and their acid addition compounds as described in U.S. Pat. No. 5,061,703 which is hereby incorporated by reference in its entireties. NMDA antagonists also include amantadine, dextromethorphan, dextrorphan, dizocilpine, ibogaine, ketamine, remacemide, and phencyclidine.

As used herein, the term “side effects associated with NMDA antagonists” refers to hallucinations, confusion, dizziness, headaches, and tiredness, experienced by subjects/patients taking an NMDA antagonist.

The term “with reduced gastrointestinal toxicity” as used herein means that the administration of the particular R-NSAID is less ulcerogenic to the gastrointestinal tract of the human or other mammal than the corresponding racemate or S-NSAID. One measure of ulcerogenic activity is the small bowel ulcer score. A rat is treated daily through oral administration of the R-NSAID for 30 days. At the end of the 30 days, the rat is sacrificed and the intestines removed. Lesions of appreciable size in the mucosa are measured. A cumulative score equaling the sum of the diameters of the ulcers measured are reported as the ulcer score. An ulcer score essentially equal to that of a control rat, or a reduction of the ulcer score of at least 50 to 90%, preferably at least 80%, as compared to the corresponding S-NSAID or racemate, is considered a reduction in gastrointestinal toxicity. In another embodiment, the term “with reduced gastrointestinal toxicity” refers the ability to administer a lower amount of NSAID (such as sulindac, flurbiprofen, ibuprofen) such that unwanted gastrointestinal toxicity side-effects are reduced.

As used herein, the term “R-NSAID” refers to the R-enantiomer of a non-steroidal anti-inflammatory drug. R-NSAIDs can be administered as substantially pure R-enantiomers or as part of a racemic mixture. In a preferred embodiment, the amount of R-NSAID is adjusted to avoid adverse effects associated with the S enantiomer of the NSAID. The term “substantially free of the (S)-stereoisomer” as used herein means that the composition contains a greater proportion of the (R)-isomer of the R-NSAID in relation to the (S)-isomer of the R-NSAID. In a preferred embodiment the term “substantially free of its (S)-stereoisomer” as used herein means that the composition contains at least 90% by weight of (R)-NSAID and 10% by weight or less of (S)-NSAID; in a more preferred embodiment at least 95% (R)-NSAID and 5% by weight or less of its (S)-isomer. These percentages are based on the total amount of NSAID present in the composition. In an embodiment, the composition for use in the invention contains approximately 99% by weight of (R)-NSAID, and 1% or less of (S)-NSAID. In another preferred embodiment, the composition for use in the invention contains greater than 99% by weight of the (R)-isomer of NSAID, again based on the total amount of NSAID present. The terms “substantially optically pure (R)-isomer of NSAID,” “optically pure (R)-isomer of NSAID,” “optically pure (R)-NSAID” and “(R)-isomer of NSAID” are also encompassed by the above-described amounts. The term “substantially free” indicates that the amount of S-NSAID, if any, present in the composition is insufficient to elicit an adverse effect in the patient to whom the composition is administered or, at most elicits an adverse effect that is tolerable to the patient and is outweighed by the beneficial effect or effects. NSAIDs include, but are not limited to 5,5-dimethyl-3-(3-fluorophenyl)-4-(4-methylsulfonyl)phenyl-2(5H)-furanone, 5,5-dimethyl-3-isopropyloxy-4-(4′-methylsulfonylphenyl)-2(5H)-furanone, resveratrol, flufemic acid, meclofenamic acid, fenoprofen, carprofen, ibuprofen, ketoprofen, sulindac, flurbiprofen, indomethacin, naproxen, etolodac, tiaprofenic, suprofen, ketorolac, pirprofen, indoprofen, benoxaprofen, oxaprozin, diflunisal, and nabumetone.

The chemical structures of NSAIDs vary. Certain NSAIDs, such as ketoprofen and flurbiprofen are arylpropionic acids, while others are cyclized derivatives of arylpropionic acids, arylacetic acids, thiazinecarboxamides, etc. Depending on the structure of a particular NSAID, the compound may or may not exhibit chirality, i.e, may not have R- and S-enantiomers

NSAID derivatives are compounds generated by modifying functional groups of known NSAIDs. For example, substitutions to the aminocarboxylic acid, arylacetic acid, and arylpropionic acid groups of NSAIDs are typically performed to produce a NSAID derivative or analogue. Modifications and additions to indole compounds are typical ways of producing NSAID analogues. For example, alkyl, hydroxyl alkyl, phenyl, benzyl, or thienyl groups may be added to indoles in various combinations in order to prepare NSAID derivatives and analogues. In addition, structural analogues of NSAIDs can be identified by commercially available computer modeling programs. In a specific example of a NSAID derivative or analogue of the present invention, at least one of the compounds is a derivative or analogue of R-flurbiprofen.

The NSAIDS may be a nitrosated or nitrosylated NSAID, NSAID derivative, or NSAID analogue instead of an R-NSAID. Nitrosation refers to linking a nitrogen monoxide group (NO) to a compound. Nitrosylation refers to linking a nitrogen dioxide group (NO₂) to a compound. Nitrosated and/or nitrosylated NSAIDs and nitrosated and/or nitrosylated NSAID derivatives are known to release nitric oxide, which may increase the efficacy of clearing Aβ deposits in an individual. (See Jantzen et al., Journal of Neuroscience, 22:2246-2254 (2002)). Examples of nitrosated and/or nitrosylated NSAIDS are found in U.S. patent application Ser. No. 09/938,560, which is incorporated herein by reference. In a specific example of this embodiment, at least one of the compounds may be nitrosated or nitrosylated flurbiprofen (or R-flurbiprofen) instead of R-flurbiprofen.

As used herein, the term “activity” refers to functions, which include processes (such as metabolism, catabolism, or enzymatic functions), movement, binding, and exerting therapeutic effects.

As used herein, the term “interactor” or “CYP2C9 interactor” refers to a compound that is capable of acting as a substrate or inhibitor of CYP2C9.

As used herein, the term “inhibitor” refers to a compound that prevents and/or slows synthesis, activation, and/or activity.

As used herein, the term “substrate” refers to the substance acted upon by an enzyme and/or other compound.

The term “compound” as used herein encompasses all types of organic or inorganic molecules, including but not limited to proteins, peptides, polysaccharides, lipids, nucleic acids, small organic molecules, inorganic compounds, and derivatives thereof.

As used herein, the term “analogue” encompasses a chemical compound that is structurally similar to another but differs slightly in composition. Such differences can be the replacement of one atom or functional group by an atom or functional group of a different element.

As used herein, the term “derivative” encompasses a chemical substance related structurally to another substance, in which the chemical substance is able to be made from the related substance.

As used herein, the term “statin” refers to a class of pharmaceuticals known as 3-hydroxy-3-methylglutaryl-Coenzyme-A reductase (HMG-CoA Reductase) inhibitors. Statins are able to inhibit HMG-CoA reductase, the rate-limiting enzyme that converts HMG-CoA into mevalonate. Statins include, but are not limited to, atorvatstatin, simvastatin, lovastatin, fluvastatin, pravastatin, cerivastatin, rosuvastatin, pitavastatin, compounds described in WO 00/96311, WO 00/28981, WO 86/07054,U.S. Pat. No. 4,647,576, U.S. Pat. No. 4,686,237, all of which are hereby incorporated by reference in their entireties.

As used herein, the term “increasing the secretion of Aβ₃₈” refers to increasing the amount of Aβ₃₈ produced from processing amyloid precursor proteins (APPs).

As used herein, the term “preventing Alzheimer's disease” refers to a slowing of the progression or of the onset of the disease or the signs or symptoms thereof. Preventing Alzheimer's disease can include stopping the onset of the disease or signs or symptoms thereof.

As used herein, “NSAIDs” refers to non-steroidal anti-inflammatory drugs. NSAIDs are distinct from steroidal drugs with anti-inflammatory properties such as corticosteroids. Typically, NSAIDs are organic acids that have analgesic (pain-reducing), anti-inflammatory, and anti-pyretic (fever-reducing) effects. Some examples of NSAIDs include salicylic acid (Aspirin), ibuprofen (Motrin, Advil), naproxen (Naprosyn), sulindac (Clinoril), diclofenac (Voltaren), piroxicam (Feldene), ketoprofen (Orudis), idflunisal (Dolobid), nabu-metone (Relafen), etodolac (Lodine), oxaprozin (Daypro), Meclofenamic acid (Meclofen) and indomethacin (Indocin). NSAIDs can be grouped into classes, for example, amino aryl carboxylic acid derivative (e.g., flufenamic acid, meclofenamic acid); aryl acetic acid derivatives (e.g., indomethacin, sulindac); and aryl propionic acid derivatives (fenoprofen, ibuporofen, carprofen).

As used herein, a “racemic mixture” includes amounts of R- and S-enantiomers sufficient to elicit an effect specific to the R- or S-enantiomer, respectively. Therefore, a racemic mixture may include equal or unequal amounts of R- and S-enantiomers. The composition of R- and S-enantiomers may have a range of 5% to greater than 95% by weight. For example, the racemic mixture may contain 95% by weight of an R-NSAID and 5% by weight of the corresponding S-NSAID, based upon the total amount of NSAID present in the composition. Thus, the ratio of R- to S-enantiomer in the composition is within the range of: 5:95, 10:90, 20:80, 30:70, 40:60, 50:50, 60:40, 70:30, 80:20, 90:10, 95:5.

As used herein, the term “substantially free of S-flurbiprofen” indicates that the amount of S-flurbiprofen, if any, present in the composition is insufficient to elicit an adverse effect in the patient to whom the composition is administered or, at most elicits an adverse effect that is tolerable to the patient and is outweighted by the beneficial effect or effects. A composition that is substantially free of S-enantiomers may contain less than 5% S-enantiomer by weight. For example, a composition substantially free of S-flurbiprofen may contain 98% by weight of R-flurbiprofen and 2% by weight of S-flurbiprofen, based upon the total amount of flurbiprofen present in the composition.

The terms “neurodegenerative diseases” and “neurodegenerative disorders” include such diseases and impairments as Alzheimer's disease, dementia, MCI, Huntington's disease, Parkinson's disease, amyotrophic lateral sclerosis, epilepsy, and Pick's disease.

As used herein, the term “pharmaceutically acceptable salts and esters” refers to salt and ester forms that are pharmacologically acceptable and substantially non-toxic to the subject being administered the composition of the present invention. Typically, a salt is formed when the hydrogen of an acid is replaced by a metal or its equivalent and an ester formed through the exchange of a replaceable hydrogen of an acid for an organic radical, usually using an alcohol or other organic compound rich in OH groups.

Pharmaceutically acceptable salts include conventional acid-addition salts or base-addition salts formed from suitable non-toxic organic or inorganic acids or inorganic bases. For example, acid-addition salts include salts derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and famaric acid. Examples of base-addition salts include salts derived from ammonium hydroxides (e.g., a quaternary ammonium hydroxide such as tetramethylammonium hydroxide), salts derived from inorganic bases such as alkali or alkaline earth-metal (e.g., sodium, potassium, lithium, calcium, or magnesium) hydroxides, and salts derived from organic bases such as amines, benzylamines, piperidines, and pyrrolidines.

The compound for use in the methods of the invention include pharmaceutically acceptable esters, salt, and biocleavable esters thereof.

3.0. Assays for COX-1/2 Activity and Aβ Levels

In vitro cellular COX inhibition can be determined using specific assays for inhibition of COX-1 and COX-2. An art-known cellular assay for determining COX inhibition is based on the production of prostaglandin-E₂ from exogenous arachidonic acid in cells expressing COX-1, COX-2, or a combination thereof. COX enzymes (prostaglandin H synthase) catalyze the rate-limiting step in prostaglandin synthesis from arachidonic acid. Cell lines are known and available that express at least one form of the enzyme. For example, a human skin fibroblast line can be induced with IL-1 to synthesize COX-2, and a kidney epithelial cell line 293 has been stably transfected to constitutively express COX-1. In these assays, arachidonic acid can be added exogenously to increase signal to readably detectable levels. Thus, the amount of prostaglandin-E₂ in the extracellular medium can be assayed by radioimmunoassay, for measuring COX activity. IC₅₀ values for compounds for COX-1 and COX-2 can be determined by an ordinary skilled artisan. Anti-inflammatory activities of compounds can be determined using the art-known rat paw edema assay.

The effects of the compositions and compounds of the invention can be determined by examining the secretion of Aβ₄₂ by a CHO cell line that expresses APP. Untreated cell cultures, cell cultures treated with a compound of the invention and a carrier, carrier treated cell cultures can be examined and compared, and Aβ₄₂ levels secretion levels can be determined.

A CHO (Chinese hamster ovary) cell line expressing APP can be culture for an appropriate amount of time and the supernatants analyzed for Aβ₄₂ and Aβ₄₀ levels using end-specific Aβ₄₂ and Aβ₄₀ ELISAs (Suzuki et al. (1994) Science 264:336-340). Supernatants from cell cultures grown in the presence of varying concentrations of the compositions of the invention and active ingredients thereof, ranging from about 0.1 μM to about 500 μM are analyzed for Aβ₄₂ and Aβ₄₀ levels. Supernatants from control cell cultures treated with carrier and receiving no treatment are also analyzed for Aβ₄₂ and Aβ₄₀ levels. Compounds and compositions which alter Aβ₄₂ levels by more than about 15% as compared to the cultures grown in the presence of carrier under similar conditions are said to lower Aβ₄₂ levels.

For further description of assays, cell line, and techniques capable of assessing COX inhibitory activity and Aβ₄₂ lowering activity see, e.g., WO 01/78721, and references cited therein, all of which are incorporated herein in their entirety.

4.0. Additional Combination Therapy

The invention further provides additional combination therapy strategies for treating neurodegenerative disorders such. It is noted that the combination treatment can be applied to a patient for purposes of treating any suitable diseases and disorders, including but not limited to, dementia, Alzheimer's disease, mild cognitive impairment (MCI), tauopathies (e.g., corticobasal degeneration, frontotemporal dementia with Parkinsonism linked to chromosome 17, and progressive supranuclear palsy), Down's Syndrome, and others. Thus, the patient treated can have one of the diseases and disorders requiring treatment, or have two or more of the diseases and disorders.

According to this aspect of the invention, an individual in need of treatment is administered an effective amount of an R-NSAID (e.g., R-flurbiprofen), at least one statin (such as atorvastatin, simvastatin, lovastatin, fluvastatin, pravastatin, cerivastatin, rosuvastatin, and pitavastatin), and at least one NMDA antagonist (such as memantine, adamantane, amantadine, an adamantane derivative, dextromethorphan, dextrorphan, dizocilpine, ibogaine, ketamine, remacemide, and phencyclidine), and optionally at least one compound selected from the group consisting of NSAIDs, COX-2 inhibitors (cyclooxygenase-2), β-secretase inhibitors, and γ-secretase inhibitors, acetylcholine esterase inhibitors, and GABA-A alpha inverse agonist (see WO 00/27382, WO 96/25948, WO 98/50385 which are herein incorporated by reference in there entireties). Preferred acetylcholine esterase inhibitors include tacrine, donepezil, rivastigmine, and galantamine. According to a preferred aspect of this embodiment the NSAID is selected from the group consisting of 5,5-dimethyl-3-(3-fluorophenyl)-4-(4-methylsulfonyl)phenyl-2(5H)-furanone, 5,5-dimethyl-3-isopropyloxy-4-(4′-methylsulfonylphenyl)-2(5H)-furanone, resveratrol, flufemic acid, meclofenamic acid, fenoprofen, carprofen, ibuprofen, ketoprofen, sulindac, flurbiprofen, indomethacin, naproxen, etolodac, tiaprofenic, suprofen, ketorolac, pirprofen, indoprofen, benoxaprofen, oxaprozin, diflunisal, and nabumetone. The combination therapy of the invention, in theory, is thought to provide a synergistic effect in reducing Aβ₄₂ levels and is thought to be especially effective for treating and preventing neurodegenerative disorders including Alzheimer's disease, dementia, and MCI. The invention further encompasses compositions comprising the combination of active ingredients of this aspect of the invention.

According to another aspect of the invention, an individual in need of treatment is administered an effective amount of two compounds that are capable of interacting with CYP2C9, wherein at least one of the compounds is an Aβ₄₂ lowering agent, and optionally at least one compound selected from the group consisting of NSAIDs, COX-2 inhibitors (cyclooxygenase-2), β-secretase inhibitors, and γ-secretase inhibitors, acetylcholine esterase inhibitors, and GABA-A alpha inverse agonist (see WO 00/27382, WO 96/25948, WO 98/50385 which are herein incorporated by reference in there entireties). Preferred acetylcholine esterase inhibitors include tacrine, donepezil, rivastigmine, and galantamine. According to a preferred aspect of this embodiment the NSAID is selected from the group consisting of 5,5-dimethyl-3-(3-fluorophenyl)-4-(4-methylsulfonyl)phenyl-2(5H)-furanone, 5,5-dimethyl-3-isopropyloxy-4-(4′-methylsulfonylphenyl)-2(5H)-furanone, resveratrol, flufemic acid, meclofenamic acid, fenoprofen, carprofen, ibuprofen, ketoprofen, sulindac, flurbiprofen, indomethacin, naproxen, etolodac, tiaprofenic, suprofen, ketorolac, pirprofen, indoprofen, benoxaprofen, oxaprozin, diflunisal, and nabumetone. In a specific example, at least one of the CYP2C9 interacting compounds is R-flurbiprofen or ibuprofen and at least one of the compounds is a statin, preferably fluvastatin or rosuvastatin.

According to another aspect of the invention, an individual in need of such treatment is administered an effective amount of R-flurbiprofen, at least one statin such as atorvastatin, simvastatin, lovastatin, fluvastatin, pravastatin, cerivastatin, rosuvastatin, and pitavastatin, and at least one NMDA antagonist such as memantine, adamantane, amantadine, an adamantane derivative, dextromethorphan, dextrorphan, dizocilpine, ibogaine, ketamine, remacemide, and phencyclidine. The treatment regime used in the combination therapy can involve administration of a composition comprising the combination of active ingredients, the concomitant administration of separate compositions, each comprising at least one active ingredient. Furthermore, the administration of the active ingredients can be performed at different times and/or different routes. For example, a composition having one active ingredient can be administered in the morning, and a composition having the other active ingredients can be administered in the evening. Another example would involve the administration of a composition having two active ingredients orally while the third active ingredient is administered intravenously.

5.0. Preparation of Compounds of the Invention

The compounds of the invention can be prepared by a variety of art known procedures. In one aspect, the R-NSAID employed in the compositions and methods disclosed herein can be selected from the group consisting of selected R-flurbiprofen, R-ibuprofen, R-ketoprofen, R-naproxen, R-tiaprofenic acid, R-suprofen, R-carprofen, R-pirprofen, R-indoprofen, and R-benoxaprofen. The R-NSAID can also be a cyclized derivative of an arylpropionic acid, such as R-ketorolac, or an arylacetic acid, such as R-etodolac. Descriptions of specific NSAIDs and their preparation can be found in various publications. Ketoprofen is described in U.S. Pat. No. 3,641,127. Flurbiprofen is described in U.S. Pat. No. 3,755,427. Ketorolac is described in U.S. Pat. No. 4,089,969. A large number of the NSAIDs useful according to the invention are commercially available either in the form of racemic mixtures or as optically pure enantiomers. In all cases racemic mixtures contain equal amounts of the R- and S-isomers of the NSAID are provided. For example, the following racemates can be obtained through Sigma Chemical Co.: ketoprofen, flurbiprofen, etodolac, suprofen, carprofen, indoprofen and benoxaprofen. Naproxen, marketed as the S-isomer only, is also available from this source. Additionally, many commercial sources exist for the stereospecific R-isomers of many NSAIDs. R-ketoprofen, R-flurbiprofen and R-ketorolac, for example, are available through Sepracor, Inc.; R-naproxen can be obtained as the sodium salt through Sigma Chemical Co.; R-etodolac is available from Wyeth-Ayerst; R-tiaprofenic acid is available through Roussel (France, Canada, Switzerland, Spain, Denmark, Italy); R-suprofen is manufactured by McNiel Pharmaceuticals; R-carprofen is available from Roche; R-pirprofen is available through Ciba (France, Belgium, Denmark); R-indoprofen can be obtained through Carlo Elba (Italy, U.K.); and R-benoxaprofen is manufactured by Eli Lilly Co (Indianapolis, Ind.).

Descriptions and preparation of specific compounds of the invention can be found in various publications. For example, ketoprofen is described in U.S. Pat. No. 3,641,127; flurbiprofen is described in U.S. Pat. No. 3,755,427, and ketorolac is described in U.S. Pat. No. 4,089,969.

In addition, many of the compounds of the present invention can be obtained commercially. For example, a large number of the NSAIDs useful according to the invention are commercially available either in the form of racemic mixtures or as optically pure enantiomers. In all cases racemic mixtures contain equal amounts of the R- and S-isomers of the NSAID are provided. For example, the following racemates can be obtained through Sigma Chemical Co.: ketoprofen, flurbiprofen, etodolac, suprofen, carprofen, indoprofen and benoxaprofen. Naproxen, marketed as the S-isomer only, is also available from this source. Additionally, many commercial sources exist for the stereospecific R-isomers of many NSAIDs. R-ketoprofen, R-flurbiprofen and R-ketorolac, for example, are available through Sepracor, Inc.; R-naproxen can be obtained as the sodium salt through Sigma Chemical Co.; R-etodolac is available from Wyeth-Ayerst; R-tiaprofenic acid is available through Roussel (France, Canada, Switzerland, Spain, Denmark, Italy); R-suprofen is manufactured by McNiel Pharmaceuticals; R-carprofen is available from Roche; R-pirprofen is available through Ciba (France, described in U.S. Pat. No. 5,177,080; fluvastatin sodium, marketed under the name LESCOL, by Novartis Pharmaceuticals, and described in U.S. Pat. No. 5,354,772. All of the patents referenced in this section are hereby incorporated by reference in their entireties.

NSAID derivatives and NSAID analogues can be obtained from Sigma, Biomol, Cayman Chemical, ICN, or from the web through the Chemnavigator website. Novel NSAID derivatives and novel NSAID analogues can be chemically synthesized using methods described in many published protocols and the starting materials for the synthesis of the compounds of the invention are available or readily preparable. In fact, there are dozens of reports presenting synthesis of novel derivatives of known NSAIDs (see Dewitt Molecular Pharmacology 55:625-631, (1999)). For example Kalgutkar et al. (2000) PNAS 97:925-930 have made derivatives of indomethacin and meclofenamic acid and Bayly et al. Biorg and Med Chem Letters 9:307-312 (1999) have made derivatives of flurbiprofen.

For example, substitutions to the aminocarboxylic acid, arylacetic acid, and arylpropionic acid groups of NSAIDs are typically performed to produce NSAID derivatives and analogues. Modifications and additions to indole compounds are also typical. For example, alkyl, hydroxyl alkyl, phenyl, benzyl, or thienyl groups may be added to indoles in various combinations in order to prepare NSAID analogues.

In a specific example, flurbiprofen, fenoprofen, and carprofen derivatives can be prepared by performing modifications including, but not limited to: (1) altering the position of the propionic acid substituent on the phenyl ring, (2) altering the position or type of substituents on the phenyl ring opposite the propionic acid substituent, (3) altering the bond connecting the two phenyl rings, (4) replacing the acetic acid substituent with a carboxylic acid substituent or other derivative.

Meclofenamic acid and flufenamic acid derivatives can be prepared by performing modifications including, but not limited to: (1) altering the position of the carboxylic acid substituent on the phenyl ring, (2) altering the position or type of substituents on the phenyl ring opposite the carboxylic acid substituent, (3) altering the bond connecting the two phenyl rings, (4) replacing the carboxylic acid substituent with a propionic acid substituent or other derivative.

Sulindac sulfide derivatives can be prepared by performing modifications including, but not limited to: (1) replacing the fluoride group with another substituent, (2) replacing the propionic acid derivative with another substituent, (3) replacing the methylthio derivative with another substituent.

Indomethacin derivatives can be prepared by performing modifications including, but not limited to: (1) substituting the carboxylic acid or indole nitrogen with another substituent.

The preparation of nitrosated and/or nitrosylated NSAIDs can be prepared by one skilled in the art in many ways and at a variety of locations. For example, NSAIDs can be nitrosated and/or nitrosylated at locations such as oxygen (hydroxyl condensation), sulfur (sulflhydryl condensation), carbon, and/or nitrogen. In a specific example, nitroxybutyl ester may be coupled to flurbiprofen through a methoxyphenyl linker. Other examples and methods of preparing nitrosated and/or nitrosylated NSAIDs can be found in U.S. patent application Ser. No. 938,560, which is incorporated herein by reference.

Memantine, adamantane, and adamantane derivatives can be prepared by any method known in the art. See, for example U.S. Pat. No. 5,061,703, which is hereby incorporated by reference.

Dextromethorphan ((+)-3-methoxy-N-methylmorphinan) is available commercially (Sigma-Aldrich, St. Louis, Mo.) and can be prepared by any method known in the art. Dextrorphan ((+)-3-hydroxy-N-methylmorphinan) can be prepared by any method known in the art (CAS-RN 297-90-5). Dizocilpine ((+)-5-methyl-10,11-dihydro-5H-di[a,d]-cyclohepten-5,10-imine) can be prepared by any method known in the art and is available commercially from Voigt Global Distribution LLC (Kansas City, Mo.) and other sources. Ibogaine (12-methoxyibogamine, NIH 10567, Endabuse) can be prepared by any method known in the art. For example, synthesis of ibogaine is described by G. Büchi et al., J. Am. Chem. Soc. 88, 3099 (1966) and P. Rosenmund et al., Chem. Ber. 180, 1871 (1975). Alternatively ibogaine can be isolated from natural sources such as Tabernanthe iboga, a shrub indigenous to Central-West Africa J. Dybovsky et al. Acad. Sci. (Paris) 133, 748 (1901). Ketamine can be prepared by any method known in the art. For example, U.S. Pat. No. 3,254,124 describes the preparation of ketamine. Remacemide (FPL 12924AA; 2-amino-N-(1-methyl-1,2-diphenylethyl) acetamide hydrochloride) can be prepared by any method known in the art. See, for example, Riley et al. Drug Metab Dispos September; 23(9):922-8 (1995) and references cited therein. Phencyclidine can be prepared by any method known in the art. For example, U.S. Pat. No. 3,097,136 describes the preparation of phencyclidine. Amantadine (Symmetrel®) is available commercial from, for example, Endo Pharmaceuticals (Chadds Ford, Pa.)

Examples of commercially available statins include, but are not limited to: lovastatin, marketed under the trademark MEVACOR by Merck, and described in U.S. Pat. No. 4,231,938; simvastatin, marketed under the trademark ZOCOR by Merck, and described in U.S. Pat. No. 4,444,784; atorvastatin calcium, marketed under the name LIPITOR by Parke-Davis, and described in U.S. Pat. No. 5,273,995; cerivastatin sodium, marketed under the name BAYCOL, by Bayer.

The present invention also includes derivatives and analogues of statins. Statin derivatives and analogues can be prepared in a variety of manners. For example, modifications and additions to indole compounds are typical. For example, alkyl, hydroxyl alkyl, phenyl, benzyl, or thienyl groups may be added to indoles in various combinations in order to prepare statin derivatives and analogues. In addition, substitutions to the aminocarboxylic acid, arylacetic acid, and arylpropionic acid groups of statins are typically performed to produce derivatives or analogues. Other examples and methods of preparing statin derivatives and analogues can be found in U.S. Pat. No. 4,739,073 and U.S. Pat. No. 5,354,772, which are incorporated herein by reference.

6.0. Formulation, Dosages, and Routes of Delivery

The compositions according to the invention are those suitable for enteral, such as oral or rectal, transdermal, topical, and parenteral administration to an individual, for the prevention and/or treatment of neurodegenerative disorders including Alzheimer's disease, dementia, and/or MCI. Such compositions can comprise an effective amount of a compound or compounds as described herein, alone or in combination, and with one or more pharmaceutically acceptable carriers.

In conjunction with another active ingredient, a compound of the invention may be administered either simultaneously, before or after the other active ingredient, either separately by the same or different route of administration or together in the same pharmaceutical formulation.

The dosage of active compound(s) administered is dependent on the body weight, age, individual condition, and on the form of administration. A unit dosage for oral administration to a mammal of about 50 to 70 kg may contain between about 0.1 mg to about 2000 mg, more preferably from about 1 to about 1000 mg of each active ingredient.

The NMDA antagonist may be administered in the form of their pharmaceutically-acceptable acid addition salts including, for example, the hydrochlorides, hydrobromides, sulfates, acetates, succinates or tartrates, or their acid addition salts with fumaric, maleic, citric, or phosphoric acids. The NMDA antagonist compounds can be administered in suitable form in doses ranging from about 0.01 to 100 mg/kg. Appropriate presentation forms are, for example, combinations of the active substance with common pharmaceutical carriers and adjuvants in the form of tablets, coated tablets, and sterile solutions or suspensions for injection. Pharmaceutically-acceptable carriers are, for example, lactose, sucrose, sorbitol, talc, stearic acid, magnesium stearate, gum arabic, corn starch, or cellulose, combined with diluents such as water, polyethylene glycol, etc. Solid presentation forms are prepared according to common methods and may contain up to 500 mg of the active ingredient per unit.

Preferred daily oral dosages for NMDA antagonists used in the methods of the invention are as follows: memantine from about 0.1 mg to about 40 mg, preferably from about 0.5 mg to about 20 mg, more preferably from about 1 mg to about 10 mg; amantadine from about 1 mg to about 600 mg, preferably from about 1 mg to about 300 mg, more preferably from about 1 mg to about 200 mg; dextromethorphan from about 1 mg to about 600 mg, preferably from about 1 mg to about 400 mg, more preferably from about 1 mg to about 200 mg; dextrorphan from about 1 mg to about 600 mg, preferably from about 1 mg to about 400 mg, more preferably from about 1 mg to about 200 mg; ketamine from about 1 mg to about 1000 mg, preferably from about 1 mg to about 500 mg, more preferably from about 1 mg to about 300 mg; dizocilpine from about 1 mg to about 200 mg, preferably from about 1 mg to about 100 mg, more preferably from about 1 mg to about 50 mg. It is readily recognized by the skilled artisan that these dosages may need to be modified depending on the route of administration, stage of the disease, and condition of the patient.

Exemplary daily dosages of R-flurbiprofen include at least 100 mg, at least 200 mg, at least 400 mg, at least 800 mg, at least 1600 mg, at least 2000 mg, and at least 2400 mg. Preferred daily dosages of R-flurbiprofen are from about 1 mg to about 2000 mg, preferably from about 1 mg to about 1600 mg, more preferably from about 1 mg to about 800 mg, and even more preferably from 1 mg to about 600 mg. In exemplary embodiments, a dosage having R-flurbiprofen in an amount of about 400 mg to about 800 mg per dose is included in the present invention. The dose can be provided twice daily, in a single or multiple dosage units (i.e., tablets or capsules) having about 300 mg R-flurbiprofen, 400 mg R-flurbiprofen, 500 mg R-flurbiprofen, 600 mg R-flurbiprofen, 700 mg R-flurbiprofen, or 800 mg R-flurbiprofen, or a pharmaceutically acceptable salt or ester thereof. For example, an exemplary dose is 400 mg of R-flurbiprofen, which may be provided in a composition of the invention comprising 400 mg R-flurbiprofen and an effective amount of NMDA antagonist and/or CYP2C9 inhibitor, and a carrier or vehicle suitable for oral administration, e.g., in tablets or capsules. Another exemplary dose is 800 mg of R-flurbiprofen, which may be provided in a composition of the invention comprising 800 mg R-flurbiprofen and an effective amount of NMDA antagonist and/or CYP2C9 inhibitor, and a carrier or vehicle suitable for oral administration, e.g., in tablets or capsules.

Preferably, the compositions are substantially free of S-flurbiprofen. In one aspect, substantially free of the S-stereoisomer means at least 90% by weight R-flurbiprofen to 10% by weight or less of S-flurbiprofen of the total flurbiprofen (S+R flurbiprofen) in said pharmaceutical composition. In another aspect, substantially free of the S-stereoisomer means at least 95% by weight R-flurbiprofen to 5% by weight or less of S-flurbiprofen of the total flurbiprofen (S+R flurbiprofen) in the pharmaceutical composition. In yet another aspect, substantially free of the S-stereoisomer means at least 99% by weight R-flurbiprofen to 1% by weight or less of S-flurbiprofen of the total flurbiprofen (S+R flurbiprofen) in the pharmaceutical composition. In yet another aspect, substantially free of the S-stereoisomer means at least 99.9% by weight R-flurbiprofen to 0.1% by weight or less of S-flurbiprofen of the total flurbiprofen (S+R flurbiprofen) in the pharmaceutical composition. In one aspect, a preferred dosage form is a tablet. In another aspect, a preferred dosage form is a capsule. In other aspects, the composition provides an improvement or lessening in decline in biochemical disease marker progression, plaque pathology, quality of life indicators or combinations of any disease parameters.

In preferred embodiments, in the R-flurbiprofen-containing compositions and combination treatment methods of the present invention, R-flurbiprofen or a pharmaceutically acceptable salt or ester thereof is administered in an amount sufficient to result in a plasma Cₘₐₓ of about 20 to about 150 μg per mL, and wherein said individual is known to have, or is suspected of having, AD or MCI. In a more specific embodiment, said plasma Cₘₐₓ is from about 30 to about 95 μg per mL. In another more specific embodiment, said Cₘₐₓ is from about 40 to about 80 μg per mL. In another embodiment, said Cₘₐₓ is between about 100 and about 600 μM. In a more specific embodiment, said plasma Cₘₐₓ is from about 150 to about 380 μM. In another more specific embodiment, said Cₘₐₓ is from about 170 to about 240 μM. In a specific, preferred embodiment, said individual has mild to moderate AD or MCI.

In another embodiment, R-flurbiprofen or a pharmaceutically acceptable salt or ester thereof is administered in an amount sufficient to result in a cerebrospinal fluid R-flurbiprofen Cₘₐₓ of about 0.05 to about 7.5 μg per mL, and wherein said individual is known to have, or is suspected of having, AD or MCI. In another embodiment, said Cₘₐₓ is from about 0.08 to about 4.5 μg per mL. In another embodiment, the R-flurbiprofen or a pharmaceutically acceptable salt or ester thereof is administered in an amount sufficient to result in a cerebrospinal fluid R-flurbiprofen Cₘₐₓ of about 2 to 30 μM; from about 3.2 μM to about 20 μM; or from about 4 μM to about 12 μM. In yet another embodiment, R-flurbiprofen or a pharmaceutically acceptable salt or ester thereof is administered in an amount sufficient to result in a cerebrospinal fluid R-flurbiprofen Cₘₐₓ of at least about 0.05, 0.10, 0.25, 0.50, 1.0, 1.5, 2.0, 2.5, 3.5, 5.0, 6.5, and/or 7.5 μg per mL.

The time to achieve plasma R-flurbiprofen Cₘₐₓ will depend upon the individual to be treated, but is preferably between 0.70 to 3.75 hours. In various embodiments, the tₘₐₓ (time to Cₘₐₓ) is from about 0.75 to 2.00 hours, or is from about 0.75 hour to about 1.75 hours. For example, tₘₐₓ can be about 2 hours after administration. Preferably, the t_{1/2} (half-life) is from about 3.75 to about 8.5 hours. In specific embodiments, the t_{1/2} (half-life) is at least about 0.70, 0.75, 1.0, 1.25, 1.5, 1.75, 2.0, 3.0, 4.0, 5.0, 6.0, 7.0, 8.0, and/or 8.5 hours.

Somewhat more time is expected to achieve a cerebrospinal fluid Cₘₐₓ; however, this Cₘₐₓ is expected to be achieved between about 1 hour and about 6 hours after administration of a dose of R-flurbiprofen according to the invention.

R-flurbiprofen levels in the plasma or in the cerebrospinal fluid may be assessed by any art-accepted method. Determination of the concentration of R-flurbiprofen in cerebrospinal fluid may be accomplished as follows. Cerebrospinal fluid containing flurbiprofen and an internal standard, for example, flurbiprofen-D₃, is mixed with mobile phase and centrifuged. The supernatant is then transferred to a 96-well block and an aliquot of extract is injected onto a Micromass Ultima LC-MS-MS equipped with an enantio-selective column. Peak area of the m/z 243→199 flurbiprofen product ion is measured against the peak area of the m/z 246→202 flurbiprofen-D₃ internal standard product ion. Quantification may be performed using a weighted (1/x²) linear least squares regression analysis for each enantiomer generated from fortified plasma standards prepared in bulk and frozen.

The plasma half-life will also depend upon the individual to be treated. Preferably, the plasma half-life of R-flurbiprofen is from about 3.75 to about 8.5 hours. In specific embodiments, the plasma half-life of R-flurbiprofen is at least 3.75, 4.5, 5.5, 6.5, 7.5, and/or 8.5 hours. Preferably, administration of a single dose to a fasting subject provides an AUC (area under curve of concentration versus time; total drug exposure) of R-flurbiprofen of from about 200 hr·μg/mL to about 600 hr·μg/mL. In specific embodiments, administration of a single dose to a fasting subject provides an AUC of R-flurbiprofen of at least about 200 hr·μg/mL, 300 hr·μg/mL, 400 hr·μg/mL, 500 hr·μg/mL, and/or 600 hr·μg/mL. Also preferably, the R-flurbiprofen in the compositions and methods of the present invention is such that in repeating administrations an AUC₁₂ (area under curve of concentration in a 12-hour window, i.e., total drug exposure in a 12-hour window) is from about 200 hr·μg/mL to about 450 hr·μg/mL. In specific embodiments the R-flurbiprofen in the compositions and methods of the present invention is such that in repeating administrations an AUC₁₂ is at least about 200 hr·μg/mL, 250 hr·μg/mL, 300 hr·μg/mL, 350 hr·μg/mL, 400 hr·μg/mL, and/or 450 hr·μg/mL. Thus, in one embodiment, a composition of the present invention is administered to an individual having one or more indications of Alzheimer's disease or MCI, to achieve a plasma concentration in said individual of R-flurbiprofen of between 30 and 95 μg per mL by no more than 3.75 hours after administration. In a specific embodiment, said plasma concentration is achieved within 1.75 hours after administration. In another specific embodiment, said plasma concentration is achieved between 0.75 hours and 3.75 hours after administration. In another specific embodiment, said plasma concentration is between 40 and 80 μg per mL. In another specific embodiment, said individual is an individual that has been diagnoses having mild to moderate Alzheimer's disease or MCI or that would be diagnosed as having mild to moderate Alzheimer's disease or MCI according to a test of cognition.

In one embodiment, the R-flurbiprofen-containing compositions of the present invention are administered for a method of administering R-flurbiprofen to an individual, wherein said R-flurbiprofen is administered in an amount sufficient to result in a plasma Cₘₐₓ of about 35 to about 50 μg per mL, and wherein said individual is known to have, or is suspected of having, AD. In a more specific embodiment, said plasma Cₘₐₓ is from about 38 to about 48 μg per mL. In another more specific embodiment, said Cₘₐₓ is from about 39 to about 46 μg per mL. In another embodiment, the invention provides for a method of administering R-flurbiprofen to an individual, wherein said R-flurbiprofen is administered in an amount sufficient to result in a plasma Cₘₐₓ of about 45 to about 58 μg per mL, and wherein said individual is known to have, or is suspected of having, AD. In a more specific embodiment, said plasma Cₘₐₓ is from about 47 to about 56 μg per mL. In a more specific embodiment, said plasma Cₘₐₓ is from about 48 to about 55 μg per mL. In a specific, preferred embodiment, said individual has mild to moderate AD. In another specific, preferred embodiment, said individual has MCI.

In another embodiment, the time to achieve plasma Cₘₐₓ will depend upon the individual to be treated, but is preferably between 0.70 to 3.00 hours. In various preferred embodiments, the tₘₐₓ (time to Cₘₐₓ) is from about 1.0 to 2.5 hours, or is from about 1.25 hour to about 2 hours, or is from about 1.40 to about 1.75 hours. Preferably, the t_{1/2} (half-life) is from about 6.00 to about 10.0 hours; from about 6.5 to about 9.5 hours; and from about 7 to about 9 hours. Preferably the AUC (area under the curve; total drug exposure) is from about 350 (hr*ug/mL) to 750 (hr*ug/mL); is from about 400 (hr*ug/mL) to 650 (hr*ug/mL); or is from about 450 (hr*ug/mL) to 700 (hr*ug/mL). In a specific, preferred embodiment, said individual has mild to moderate AD. In another specific, preferred embodiment, said individual has MCI.

In yet another embodiment, the time to achieve plasma Cₘₐₓ will depend upon the individual to be treated, but is preferably between 0.25 to 2.00 hours. In various preferred embodiments, the tₘₐₓ (time to Cₘₐₓ) is from about 0.25 to 1.75 hours, or is from about 0.50 hour to about 1.75 hours, or is from about 0.5 to about 1.25 hours. Preferably, the t_{1/2} (half-life) is from about 3.5 to about 8.5 hours; more preferably from about 4.0 to about 8.0 hours; and more preferably from about 4.8 to about 7.5 hours. Preferably the AUC (area under the curve; total drug exposure) is from about 250 (hr*ug/mL) to 700 (hr*ug/mL); is from about 300 (hr*ug/mL) to 650 (hr*ug/mL); or is from about 350 (hr*ug/mL) to 600 (hr*ug/mL). In a specific, preferred embodiment, said individual has mild to moderate AD. In another specific, preferred embodiment, said individual has MCI.

Preferably, the daily dosage of statin is as follows: from about 1 mg to 100 mg atorvastatin; from about 0.5 mg to about 100 mg of simvastatin; from about 1 mg to 100 mg of lovastatin; from about 1 mg to about 100 mg of fluvastatin; from about 0.5 mg to about 50 mg pravastatin; from about 0.01 mg to about 1.5 mg cerivastatin; from about 1 mg to about 50 mg rosuvastatin; and from about 1 mg to about 100 mg of pitavastatin. More preferably, the daily dosage of statin is as follows: from about 1 mg to 50 mg atorvastatin; from about 0.5 mg to about 50 mg of simvastatin; from about 1 mg to 50 mg of lovastatin; from about 1 mg to about 50 mg of fluvastatin; from about 0.5 mg to about 30 mg pravastatin; from about 0.01 mg to about 0.5 mg cerivastatin; from about 1 mg to about 30 mg rosuvastatin; and from about 1 mg to about 50 mg of pitavastatin. Even more preferably, the daily dosage of statin is as follows: from about 1 mg to 25 mg atorvastatin; from about 0.5 mg to about 25 mg of simvastatin; from about 1 mg to 25 mg of lovastatin; from about 1 mg to about 25 mg of fluvastatin; from about 0.5 mg to about 15 mg pravastatin; from about 0.01 mg to about 0.25 mg cerivastatin; from about 1 mg to about 20 mg rosuvastatin; and from about 1 mg to about 25 mg of pitavastatin. Preferably, the amount of statin administered is a cholesterol lowering effective amount. A “cholesterol lowering effective amount” refers to an amount that reduces cholesterol in vivo in a human. Preferably, cholesterol, particularly LDL cholesterol is lowered by at least 5%, more preferably at least 10%, even more preferably 15%, as compared to the amount of cholesterol present in the subject in the absence of treatment.

The pharmacologically active compound(s) of the invention can be manufactured as a pharmaceutical composition comprising an effective amount of the compound(s) in conjunction or admixture with excipients or carriers suitable for either enteral or parenteral application (including, but not limited to, intravenous, intramuscular and subcutaneous routes.) Preferred are tablets and gelatin capsules comprising the active ingredient together with a) diluents, e.g. lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders e.g., magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 75%, preferably about 1 to 50%, of the active ingredient. Tablets may be either film coated or enteric coated according to methods known in the art.

Suitable formulations for transdermal application include an effective amount of a compound(s) of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

The compositions of the present invention can be prepared in any desired form, for example, tablets, powders, capsules, suspensions, solutions, elixirs, and aerosols. Carriers such as starches, sugars, microcrystalline cellulose, diluents, granulating agents, lubricants, binders, disintegrating agents, and the like may be used in the cases of oral solid preparations. Oral solid preparations (such as powders, capsules, and tablets) are preferred over oral liquid preparations. The most preferred oral solid preparations are tablets. If desired, tablets may be coated by standard aqueous or non-aqueous techniques.

In addition to the common dosage forms set out above, the compounds of the present invention may also be administered by controlled release means and/or delivery devices such as those described in U.S. Pat. Nos. 3,845,770; 3,916,899; 3,536,809; 3,598,123; and 4,008,719, the disclosures of which are hereby incorporated by reference in their entireties.

Pharmaceutical compositions of the present invention suitable for oral administration may be presented as discrete units such as capsules, cachets, or tablets, or aerosol sprays, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the conventional methods of pharmacy, but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally, with one or more additional ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent. Desirably, each tablet contains from about 0.5 mg to about 2000 mg of the active ingredient(s), and each cachet or capsule contains from about 0.5 mg to about 1000 mg of the active ingredient.

Suitable formulations for topical application, e.g. to the skin and eyes, include aqueous solutions, suspensions, ointments, creams, gels or sprayable formulations, for example, for delivery by aerosol or the like. They are thus particularly suited for use in topical, including cosmetic, formulations well-known in the art. Such may contain solubilizers, stabilizers, tonicity enhancing agents, buffers and preservatives. Formulations suitable for topical application can be prepared e.g. as described in U.S. Pat. No. 4,784,808. Formulations for ocular administration can be prepared, e.g., as described in U.S. Pat. Nos. 4,829,088 and 4,960,799.

### 7.0. EXAMPLES

### 7.1. Example 1

### Aβ Secretion Assay

To test compounds and compositions capable of modulating Aβ levels, H4 neuroglioma cells expressing APP695NL and CHO cells stably expressing wild-type human APP751 and human mutant presenilin 1 (PS1) M146L are used. Generation and culture of these cells have been described. See Murphy et al., J. Biol. Chem., 274(17):11914-11923 (1999); Murphy et al., J. Biol. Chem., 275(34):26277-26284 (2000). To minimize toxic effects of the compositions and compounds, the H4 cells are incubated for 6 hours in the presence of the various compositions and compounds. To evaluate the potential for toxic effects of the compositions and compounds, additional aliquots of cells are incubated in parallel with each composition or compound. The supernatants are analyzed for the presence of lactate dehydrogenase (LDH) as a measure of cellular toxicity.

After incubating the cells with the compositions and compounds for a pre-determined time period, sandwich enzyme-linked immunosorbent assay (ELISA) is employed to measure secreted Aβ (Aβ42 and/or Aβ40) levels as described previously. Murphy et al., J. Biol. Chem., 275(34):26277-26284 (2000). For cell culture studies serum free media samples are collected following 6-12 hours of conditioning, Complete Protease Inhibitor Cocktail added (PIC; Roche), and total Aβ concentration measured by 3160/BA27 sandwich ELISA for Aβ40 and 3160/BC05 sandwich ELISA for Aβ42. All measurements are performed in triplicate. Antibody 3160 is an affinity purified polyclonal antibody raised against Aβ1-40. HRP conjugated monoclonal antibodies BA27 for detection of Aβ40 and BC05 for detection of Aβ42 have been previously described. Suzuki et al., Science, 264(5163):1336-1340 (1994).

### 7.2. Example 2

### Determination of COX Inhibition Activity

In vitro cellular COX inhibition can be determined using specific assays for inhibition of COX-1 and COX-2 (Kalgutkar et al. J Med. Chem., 43:2860-2870 (2000)). Another art-known cellular assay for determining COX inhibition is based on the production of prostaglandin-E₂ from exogenous arachidonic acid in cells expressing COX-1, COX-2, or a combination thereof. COX enzymes (prostaglandin H synthase) catalyze the rate-limiting step in prostaglandin synthesis from arachidonic acid. Cell lines are known and available that express at least one form of the enzyme. For example, a human skin fibroblast line can be induced with IL-1 to synthesize COX-2, and a kidney epithelial cell line 293 has been stably transfected to constitutively express COX-1. In these assays, arachidonic acid can be added exogenously to increase signal to readably detectable levels. Thus, the amount of prostaglandin-E₂ in the extracellular medium can be assayed by radioimmunoassay, for measuring COX activity. IC₅₀ values for compounds for COX-1 and COX-2 can be determined by an ordinary skilled artisan. Anti-inflammatory activities of compounds can be determined using the art-known rat/mouse paw edema assay as described in Penning et al. J. Med. Chem., 40:1347-1365 (1997).

For a further description of assays, cell line, and techniques capable of assessing COX inhibitory activity and Aβ₄₂ lowering activity see, e.g., WO 01/78721, and references cited therein, all of which are incorporated herein in their entirety.

### 7.3. Example 3

### Aβ Alzheimer's Assays

The levels of the Aβ peptide can be measured in conditioned medium and in lysates from cultured neuroblastoma cells transfected with an APP expression vector (Proc. Nat Acad. Sci. USA 93:13170 (1996)). Neuronal survival and protection can be assessed with cultured neuronal cells challenged with neurotoxic factors such as the Aβ42 peptide. At various time points, cell death or viability is measured by apoptotic assay or cell counting (J. Neurobiol. 25:585, (1994); Brain Res. 706:328 (1996)). Neurite extension can be assessed with neuronal cells that are seeded in culture and the number and length of neurites that form after 16 to 20 hrs are recorded (J. Neurobiol. 25:585 (1994); J. Neurosci. 14:5461, (1994)).

### 7.4 Example 4

### NMDA Antagonist Assays

7.4.1 TCP Binding Displacement Assay:

Phencyclidine (PCP), a known NMDA antagonist, binds to the NMDA receptor-associated ionic channel and blocks ionic transport (Garthwaite et al., Neurosci. Lett. 83: 241-246 (1987)). Additionally, PCP has been shown to prevent the destruction of brain cells after cerebral ischemia in rats (Sauer et al., Neurosci. Lett. 91:327-332 (1988)).

The interaction between NMDA antagonists and the PCP bond is examined as follows. A membrane preparation of rat cortex is incubated with ³H-TCP which is an analogue of phencyclidine (PCP) (Quirion et al. Eur. J. Pharmacol. 83:155 (1982)). The interaction with the TCP binding is assessed for test compounds (i.e., 1-amino-3,5-dimethyl adamantane) in a competitive experiment. This test shows how effective test compound are in displacing TCP from the receptor. IC₅₀ values for test compounds of 100 micromolar or less, are desirable. More desirable are those test compounds having an IC₅₀ values of less than 1 micromolar.

7.4.2 NMDA Receptor Channels Blocking Assay

The following test examines whether test compounds are as effective as PCP in blocking the NMDA receptor channel. Current flowing through NMDA-activated membrane channels of cultivated spinal marrow neurons (mouse) can be measured in patch-clamp experiment (Hamill et al., Pflugers Arch. 312:85-100(1981)). After application of 20 μM NMDA, the current signal of the cell is integrated for 20 sec and recorded as a control answer (A_{c}). During succeeding application of 20 μM NMDA and 6 μM of a test compound (i.e., adamantane derivative) the intensity of the substance effect can be determined as a relative change of the control answer (A/A_{c}-A=test answer).

### 7.5 Example 5

### CYP2C9 Substrate Assay

Substrates of CYP2C9 can be identified by contacting CYP2C9-expressing cell lines with a test compound and after incubation detecting metabolites of the administered test compound. CYP2C9-expressing cell lines may be purchased or prepared by standard recombinant techniques known in the art. In this example, CYP2C9-expressing cell lines are cultured at 37° C. and 5% CO₂ in DMEM containing 10% FCS. Cells are plated at a density of 30,000 cell/well and the following day the cells are washed with PBS and incubated with the test compound. The incubation is performed in 100 microliters Ultraculture, available from BioWhittaker of Walkersville, Md. 10 microliters of supernatant are directly injected into the liquid chromatography (LC) system (e.g. model 600-MS solvent delivery system, Waters Corp.) and the compounds are separated on an Inertsil ODS-3 column (5 microm particles, 10×3.0 mm i.d., Chrompack) using gradient elution (10% to 90% mobile phase B mixed with mobile phase A in 15 minutes, A is 10% methanol/5 mM ammonium acetate and B is 95% methanol/20 mM ammoniumacetate). Mass spectrometry/mass spectrometry (MS/MS) detection is performed using a TSQ 7000 (Finnegan Mat, San Jose) that is equipped with an APCI interface by using the Instrument Control Language (ICL) to program the acquisition by Selected Reaction Monitoring. Detection of metabolites of the test compound indicates that the test compound is a substrate of CYP2C9.

### 7.6 Example 6

### CYP2C9 Inhibitor Assay

Inhibitors of CYP2C9 may be detected using commercially available kits. CYP2C9 inhibitor kits are available from companies such as Promega Corporation of Wisconsin, and BD Biosciences of California. In this example, the CYP2C9/MFC kit available from BD Biosciences of San Jose, Calif. (Catalog No. 459300 (Old HTS-3000)) is used to detect inhibitors of CYP2C9. The instructions of the kit are followed. The instructions protocol includes performing an IC₅₀ assay with one or more test compounds and a positive control inhibitor sulfaphenazole. Serial dilution of test compounds and the positive control is performed and the enzyme substrate mix is used to initiated reaction and termination. A standard curve is prepared and inhibition of test compounds is compared to the inhibition of the known CYP2C9 inhibitor sulfaphenazole.

### 7.7 Example 7

### HMG-CoA Reductase Inhibition Assay

HMG-CoA reductase activity can be measured using the method described by Edwards et al., J. Lipid Res. 1979, 20, 40-46, or the modification of this method described in U.S. Pat. No. 6,355,810, both of which are incorporated herein by reference. In this example, rat hepatic microsomes are used and the enzyme activity of HMG-CoA reductase is determined by measuring the conversion of the ¹⁴C-HMG-CoA substrate to ¹⁴C-mevalonic acid. Livers are removed from Sprague-Dawley rats and homogenized in phosphate buffer A (potassium phosphate, 0.04 M. pH 7.2; KCl, 0.05 M; sucrose, 0.1 M; EDTA, 0.03 M, aprotinin, 500 KI unitws/mL). The homogenate is spun at 16000 g for 15 minutes at 4° C. and the supernatant is removed. The supernatant is recentrifuged at 100,000 g for 70 minutes at 4° C. and pelleted microsomes are resuspended in 3-5 mL per liver of buffer A. 10 mM dithiothreitol is added and the preparation is frozen in acetone/dry ice and stored at −80° C.

The reductase activity of the HMG-CoA enzyme is assayed in 0.25 mL volume containing 0.04 M potassium phosphate, ph &.2; 0.05 M KCl, 0.10 M sucrose; 0.03 M EDTA; 0.01 M dithiothreitol; 3.5 mM NaCl; 1% dimethyl sulfoxide; 50-200 micrograms of microsomal protein; 100 microM of ¹⁴C-[D,L]-HMG-CoA (0.05 microCi, 30-60 mCi/mmol.); 2.7 mM NADPH. The reaction mixture is incubated at 37° C. for 20 minutes and the conversion of HMG-CoA substrate to the mevalonic acid product is measured. The inhibition of activity by test compounds is measured by incubating the enzymes and test compounds in the presence of NADPH. The mixture is further incubated for 15 minutes at 37° C. and the enzyme assay is initiated by adding ¹⁴C-HMG-CoA substrate. The reaction is stopped after 20 minutes by adding 25 microliters of 33% KOH. ³H-mevalonic acid (0.05 microCi) is added and the reaction mixture is incubated for 30 minutes at room temperature. The reaction mixtures are layered onto 2 G of AG 1-X8 anion exchange resin (Biorad, formate form) and poured in 0.7 cm (i.d.) glass columns and eluted with 2.5 mL of H₂O. The first 0.5 mL is discarded and the next 2.0 mL is collected and counted for both tritium and carbon-14 in 10.0 mL of Opti-fluor (Packard) scintillation fluid to determine the inhibitory effect of the test compound on the conversion of HMG-CoA substrate to mevalonic acid.

### 7.8 Example 8

### Combination Formulation

### Tablets

1 tablet contains:

| | | |
|---|---|---|
| Active NMDA ingredient | 10.0 | mg |
| (i.e., 1-amino-3,5-dimethyl adamantine) | | |
| Active NSAID ingredient | 600 | mg |
| (i.e., R-flurbiprofen) | | |
| Lactose | 300 | mg |
| Microcrystalline cellulose | 180 | mg |
| Talc | 45 | mg |
| Total | 1135 | mg |

The substances are mixed and the mixture compressed into 1135-mg tablets in a direct tableting procedure without granulation. Alternatively, the components can be mixed and divided into two 567.5 mg tablets. Such tablets or similar co-formulations can be used according to following treatments as described in Examples 6 and 7.

### 7.9 Example 9

### Oral Pharmaceutical Compositions

Oral pharmaceutical compositions of CYP2C9 interactors of the present invention can be prepared in tablet and gelatin capsule form. One formulation of oral tablets is performed by mixing 200 mg of R-flurbiprofen and 10 mg of fluvastatin with 100 mg lactose. A suitable amount of water for drying is added and the mixture is dried. The mixture is then blended with 76 mg starch, 8 mg hydrogenated vegetable oil, and 8 mg polyvinylpyrrolidinon. The resulting granules are compressed into tablets. Tablets of varying strengths are prepared by altering the ratio of CYP2C9 interactors invention in the mixture or changing the total weight of the tablet.

Another formulation of oral tablets is performed by mixing 200 mg of R-flurbiprofen and 20 mg rosuvastatin with 200 mg lactose. A suitable amount of water for drying is added and the mixture is dried. The mixture is then blended with 80 mg starch, 10 mg hydrogenated vegetable oil, and 10 mg polyvinylpyrrolidinon. The resulting granules are compressed into tablets. Tablets of varying strengths are prepared by altering the ratio of CYP2C9 interactors in the mixture or changing the total weight of the tablet.

A formulation of gelatin capsules can be prepared by mixing 400 mg of R-flurbiprofen and 30 mg of fluvastatin with 200 mg of microcrystalline cellulose and 50 mg of corn starch. 25 mg of magnesium stearate is then blended into the mixture and the resulting blend is encapsulated into a gelatin capsule. Doses of varying strengths can be prepared by altering the ratio of CYP2C9 interactors to pharmaceutically acceptable carriers or changing the size of the capsule.

Another formulation of gelatin capsules can be prepared by mixing 400 mg of R-flurbiprofen and 20 mg of fluvastatin with 100 mg of microcrystalline cellulose and 25 mg of corn starch. 50 mg of magnesium stearate is then blended into the mixture and the resulting blend is encapsulated into a gelatin capsule. Doses of varying strengths can be prepared by altering the ratio of CYP2C9 interactors to pharmaceutically acceptable carriers or changing the size of the capsule.

### 7.10 Example 10

### Treatment of Alzheimer's Disease with a R-NSAID and a NMDA Antagonist

The NMDA antagonist (i.e., 1-amino-3,5-dimethyl adamantane) can be orally administered as tablets containing 10 mg of the NMDA antagonist. These dosages can also be divided or modified, and taken with or without food. A typical dosing regime is as follows: 1st week: 5 mg once a day; 2nd week: 10 mg (5 mg twice a day) 3rd week: 15 mg (10 mg in the morning, 5 mg in the afternoon) 4th week onwards: 20 mg (10 mg twice a day). Typically, the NMDA antagonist is administered to patients having mild-to-moderate and moderate-to-severe Alzheimer's disease.

The R-NSAID (i.e., R-flurbiprofen) can be administered in liquid or solid dosage forms (preferably tablets or capsules). The dosages preferably contain 300-900 mg of active ingredient and are given twice-a-day. The dosages can also be divided or modified, and taken with or without food. The doses can be taken during treatment with the NMDA antagonist. For example, the R-NSAID can be administered in the morning as a tablet containing 800 mg of active ingredient (or two tablets each containing 400 mg of R-flurbiprofen) and the NMDA antagonist can be administered in the evening as a tablet containing 10 mg of active ingredient (i.e., 1-amino-3,5-dimethyl adamantane.)

Depending on the stage of the disease, the R-NSAID (i.e., R-flurbiprofen) can also be administered in liquid or tablet dosage forms containing lower amounts of active ingredient (i.e., 800 mg, 600 mg, 400 mg, 300 mg, 200 mg, and 100 mg.). Again, the dosages can also be divided or modified, and taken with or without food. The doses can be taken during treatment with the NMDA antagonist. For example, the R-NSAID can be administered in the morning and evening as a tablet containing 400 mg of active ingredient (i.e., R-flurbiprofen) and the NMDA antagonist can be administered in the morning and evening as a tablet containing 5 mg of active ingredient (i.e., 1-amino-3,5-dimethyl adamantane). It may be desirable to lower the amount of NMDA antagonist and R-NSAID to avoid adverse side effects associated with higher doses of these compounds. Alternatively, the NMDA antagonist and NSAID can be co-formulated into a single dosage form, i.e., liquid, tablet, capsule, etc.

### 7.11. Example 11

### Treating Neurodegenerative Disorders with an R-NSAID and a Statin

Neurodegenerative disorders such as Alzheimer's disease can be treated by administering to an individual in need a therapeutically or prophylactically effective amount of CYP2C9 interactors of the present invention. One method of treating neurodegenerative disorders involves administering 200 mg of R-flurbiprofen and 200 mg of fluvastatin daily to an individual in need. Through the administration of R-flurbiprofen and fluvastatin, an individual in need will have slowed or stopped the progressive decline of cognitive functions. The slowing or stopping of the decline of cognitive functions can be determined through measuring the loss of declarative and procedural memory, the decrease in learning ability, the reduction in attention span, and the impairment in thinking ability, judgment, and decision making.

### 7.12 Example 12

### Preventive Treatment of Alzheimer's Disease

Prior to the onset of symptoms of Alzheimer's disease or just at the very beginning stages of the disease, patients desiring prophylaxis against Alzheimer's disease can be treated with a combination of R-NSAID and NMDA antagonist. Those needing prophylaxis can be assessed by monitoring assayable disease markers, detection of genes conferring a predisposition to the disease, other risks factors such as age, diet, other disease conditions associated with Alzheimer's. Typically, the patient can be treated with a combination of NMDA antagonist and R-NSAID to delay or prevent the onset of Alzheimer's disease or symptoms thereof.

The patient desiring prophylaxis against Alzheimer's disease or prophylaxis of a worsen of the symptoms of Alzheimer's disease can be treated with a combination of R-NSAID and NMDA antagonist sufficient to delay the onset or progression of symptoms of Alzheimer's disease. For example, a patient can be treated with 800 mg of R-NSAID (i.e., R-flurbiprofen) per day and 5 mg of NMDA antagonist (i.e., 1-amino-3,5-dimethyl adamantane) per day. These amounts of these active ingredients can be modified to lessen side-effects and/or produce the most therapeutic benefit. For example, 400 mg of R-NSAID per day and 1 mg of NMDA antagonist per day can be administered to reduce sides-effects associated with the use of higher levels of the active ingredients. The R-NSAID or NMDA antagonists can be administered on different days. The preventive treatment can also be, e.g., treatment with R-NSAID for one week followed by treatment with NMDA antagonist for one week, treatment with R-NSAID for a month, followed by treatment with NMDA antagonist for one month, and the such.

### 7.13. Example 13

### Preparation of 4′-hydroxyflurbiprofen

175 g of 4-iodoanisol, 160 g of 4′-bromo-3′-nitroacetophenone, and 140 g copper powder is mixed and gradually heated for 5 hours at 80° C. and for 4 hours with a gradual raise in temperature from 80° C. to 110° C. Methylene dichloride is removed from the mixture upon cooling by evaporation. The remaining compound is 4-acetyl-4′-methoxy-2-nitrobiphenyl, which is then slowly added for 45 minutes to a solution of 300 g stannous chloride, 400 mL hydrochloric acid, and 600 mL ethanol. The resulting solution is refluxed for 3 hours and the ethanol is removed by evaporation. The remaining mixture is added to a solution of 560 g sodium hydroxide in water and ice to form a solid product. The solid product is extracted into methylene dichloride, dried over ahydrous sodium sulphate, evaporated, and recrystallized with ethanol to form 4-acetyl-2-amino-4′-methoxybiphenyl.

10 g of 4-acetyl-2-amino-4′-methoxybiphenyl is added to a mixture of 28 mL tetrahydrofuran, 10 mL water, and 40 mL hydrofluoroboric acid (42% acid by volume). The remaining solution is added to 3 g of sodium nitrite in water at a reaction temperature of 5° C. After stirring for 20 minutes, diazonium fluroborate is removed by filtration and washed with hydrofluoroboric acid and methanol/ether. Diazonium fluroborate is suspended in xylene and heated until decomposition takes place at 70° C. The mixture is then refluxed for 45 minutes and hot benzene is used to extract the residue after removing xylene by distillation. Aqueous sodium carbonate and water are used sequentially to wash the extract, and recrystallization with ethanol gives 4-acetyl-2-fluoro-4′-methoxybiphenyl.

4 g of 4-acetyl-2-fluoro-4′-methoxybiphenyl is added to a solution of 0.75 g sodium in 45 mL isopropanol. The solution is stirred, cooled to 5° C., and ethyl chloroacetate is added dropwise. After 5 hours of stirring, the solution is kept overnight at room temperature. Isopropanol is removed form the solution by evaporation and the resulting mixture is refluxed for 45 minutes with a mixture of 1.8 mL 18N aqueous sodium hydroxide and 25 mL 10% (by volume) aqueous ethanol. Ethanol is removed by distillation and the resulting mixture is diluted to 200 mL. 8.75 g of sodium metabisulphite is added to the solution and the resultant mixture is heated for 6 hours. After cooling, 20 mL of ether and 3 mL 18N sodium hydroxide are added to the solution. The layers of the solution are separated and the ethereal layer is washed with dilute acetic acid and water. The residue is dried over anhydrous sodium carbonate, evaporated, and distilled to give 2-2-fluro-4′-methoxy-4-biphenylyl)propionaldehyde. 2.5 g of 2-2-fluro-4′-methoxy-4-biphenylyl)propionaldehyde is added to 10 mL of ethanol. The mixture is added to a solution of 2 g sodium acetate and 1 g hydroxylamine sulphate in 10 mL of water. After stirring for 2 hours, refluxing for 5 minutes and collilng with ice, the solid oxime is collected from the solution by filtration and washed with ethanol. 2.5 g of the solid oxime is mixed with 55 mg nickel sulphate and 15 mL water. After heating the mixture to a boil, 2 mL 18N sodium hydroxide and 2 mL water are added and the resulting mixture is refluxed for 24 hours. Upon cooling to room temperature, dilute hydrochloric acid is added to precipitated an acid that is extracted into ether. The resulting ethereal extract is removed with aqueous potassium carbonate and the precipitated extract is then re-extracted into ether. The ethereal extract is washed with water, dried with anhydrous sodium sulphate, and evaporated to form 2-(2-fluro-4′-methoxy-4-biphenylyl)propionic acid after recrystallization from 1:1 benzene/light petroleum. 0.4 g of 2-(2-fluro-4′-methoxy-4-biphenylyl)propionic acid is mixed with 9 mL 50% acid (by volume) hydrobromic acid and 3 mL glacial acetic acid. After refluxing for 3 hours, the mixture is cooled and the remaining solid product is separated, washed with water and dried at 100° C. to give 4′-hydroxyflurbiprofen (also referred to as 2-(2-fluro-4′-hydroxy-4biphenylyl)propionic acid).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

1. A pharmaceutical composition comprising an R-NSAID and a NMDA antagonist.

2. The composition of claim 1 wherein said R-NSAID is selected from the group consisting of R-flurbiprofen, R-ketoprofen, R-ketorolac, R-naproxen, R-tiaprofenic acid, R-suprofen, R-carprofen, R-pirprofen, R-indoprofen, R-benoxaprofen, and R-etolodac.

3. The composition of claim 2 wherein said NMDA antagonist is selected from the group consisting of memantine, adamantane, amantadine, an adamantane derivative, dextromethorphan, dextrorphan, dizocilpine, ibogaine, ketamine, remacemide, and phencyclidine.

4. The composition of claim 3 wherein said R-NSAID is R-flurbiprofen.

5. The composition of claim 3 wherein said NMDA antagonist is memantine.

6. The composition of claim 4, comprising an amount of R-flurbiprofen or pharmaceutically acceptable salt or ester thereof when administered in a single dose to a fasting individual sufficient to produce a plasma Cₘₐₓ of about 25-150 μg per mL per dose.

7. A method of treating Alzheimer's disease comprising (a) identifying a patient having Alzheimer's disease and (b) administering to said patient an Alzheimer's disease treating effective amount of the composition of claim 1.

8. The method of claim 7 wherein said R-NSAID is R-flurbiprofen.

9. The method of claim 7 wherein said NMDA antagonist is memantine.

10. The method of claim 7 further comprising administration of an acetylcholine esterase inhibitor.

11. The method of claim 8 wherein said Alzheimer's disease treating effective amount of R-flurbiprofen is from about 50 mg to about 1800 mg per day and the Alzheimer's disease treating effective amount of memantine is from about 0.5 mg to about 30 mg per day.

12. A composition comprising two compounds that are capable of interacting with CYP2C9, wherein at least one of said compounds is an Aβ₄₂ lowering agent.

13. The composition of claim 12, comprising:
flurbiprofen or a derivative of flurbiprofen selected from the group consisting of a nitrosated flurbiprofen, a nitrosylated flurbiprofen, 2-(2′-fluro-4-biphenylyl)propionic acid, a nitrosated or nitrosylated 2-(2′-fluro-4-biphenylyl)propionic acid, 2-(2,2′-difluroro-4-biphenylyl)propionic acid, a nitrosated or nitrosylated 2-(2,2′-difluroro-4-biphenylyl)propionic acid, and pharmaceutically acceptable salts and ester thereof; and
an interactor of CYP2C9 selected from the group consisting of diclofenac, ibuprofen, meloxicam, naproxen, piroxicam, suprofen, celicoxib, tolbutamide, glipizide, losartan, irbesartan, amitriptyline, fluoxetine, fluvastatin, phenyloin, rosiglitazone, tamoxifen, torsemide, S-warfarin, naphthalene, amiodarone, atorvastatin, cerivastatin, fluconazole, fluvastatin, fluvoxamine, isoniazid, lovastatin, paroxetine, phenylbutazone, probenicid, sertraline, simvastatin, sulfamethoxazole, sulfaphenazole, sulphinpyrazone, teniposide, trimethoprim, zafirlukast, and rosuvastatin, and pharmaceutically acceptable salts or esters thereof.

14. The composition of claim 12 comprising:
R-flurbiprofen or a pharmaceutically acceptable salt or ester thereof; and
a statin that is an interactor of CYP2C9 or a pharmaceutically acceptable salt or ester thereof.

15. The composition of claim 14, wherein said statin is fluvastatin or rosuvastatin, or a pharmaceutically acceptable salt or ester thereof.

16. The composition of claim 15, comprising an amount of from about 1 mg to about 1600 mg of R-flurbiprofen or pharmaceutically acceptable salt or ester thereof.

17. A method of treating Alzheimer's disease comprising (a) identifying a patient having Alzheimer's disease and (b) administering to said patient an Alzheimer's disease treating effective amount of the composition of claim 12.

18. The method of claim 17 wherein said composition comprises an Alzheimer's disease treating effective amount of:
R-flurbiprofen or a pharmaceutically acceptable salt or ester thereof, and
a statin that is an interactor of CYP2C9 or a pharmaceutically acceptable salt or ester thereof.

19. The method of claim 17 further comprising administration of an acetylcholine esterase inhibitor.

20. The method of claim 18 wherein said an Alzheimer's disease treating effective amount of R-flurbiprofen is from about 1 mg to about 1600 mg per day.
